(19) United States
(12) Patent Application Publication
(10) Pub. No.: US 2021/0015529 A1
(43) Pub. Date: Jan. 21, 2021

(54) METHOD AND APPARATUS FOR THE REFIXATION OF PROSTHETIC IMPLANTS TO BONE TISSUE

(71) Applicants: Paul V. Fenton, Jr., Marblehead, MA (US); George A. Adaniya, Rockport, MA (US); Andrew Sennett, Hanover, MA (US)

(72) Inventors: Paul V. Fenton, Jr., Marblehead, MA (US); George A. Adaniya, Rockport, MA (US); Andrew Sennett, Hanover, MA (US)

(21) Appl. No.: 16/981,563

(22) PCT Filed: Mar. 18, 2019
(86) PCT No.: PCT/US19/22804

§ 371 Date: Sep. 16, 2020

Related U.S. Application Data
(63) Continuation-in-part of application No. 16/212,874, filed on Dec. 7, 2018.
(60) Provisional application No. 62/643,944, filed on Mar. 16, 2018.
(60) Provisional application No. 62/699,915, filed on Jul. 18, 2018.

(51) Int. Cl. A61B 17/88; A61B 17/32

(52) U.S. Cl. CPC A61B 17/8805; A61B 2017/320048; A61B 17/32; A61B 17/8847

(57) ABSTRACT

This invention comprises a novel approach for the refixation of a loosened implant to bone, wherein the novel approach comprises providing access to a boundary region between the implant and the bone; removing abnormal interface tissue, wear debris and/or bone cement debris from the boundary region; and inserting bone fixation material into the boundary region so that the bone fixation material engages the implant and the bone and thereby effects refixation of the loosened implant to the bone.

### REFERENCE TO PENDING PRIOR PATENT APPLICATIONS

This patent application:

(i) is a continuation-in-part of pending prior U.S. Non-Provisional patent application Ser. No. 16/212,874, filed Dec. 7, 2018 by Paul V. Fenton Jr. et al. for METHOD AND INJECTION SYSTEM FOR BONE TISSUE IMPLANT (Attorney's Docket No. PINVIVO-1), which patent application claims benefit of:

(a) prior U.S. Provisional Patent Application Ser. No. 62/595,615, filed Dec. 7, 2017 by Paul V. Fenton Jr. et al. for METHOD AND INJECTION SYSTEM FOR BONE TISSUE IMPLANT (Attorney's Docket No. PINVIVO-1 PROV);

(ii) claims benefit of pending prior U.S. Provisional Patent Application Ser. No. 62/643,944, filed Mar. 16, 2018 by Paul V. Fenton Jr. et al. for METHODS AND SYSTEM FOR REFIXATION OF BONE TISSUE TO PROSTHETIC IMPLANTS (Attorney's Docket No. PINVIVO-2 PROV); and

(iii) claims benefit of pending prior U.S. Provisional Patent Application Ser. No. 62/699,915, filed Jul. 18, 2018 by Andrew Sennett for METHODS AND SYSTEM FOR REFIXATION OF PROSTHETIC IMPLANTS TO BONE TISSUE (Attorney's Docket No. PINVIVO-3 PROV).

The four (4) above-identified patent applications are hereby incorporated herein by reference.

### FIELD OF THE INVENTION

This invention relates to a method and apparatus for percutaneous treatment of osteolysis and related pathology in the periprosthetic region adjacent to painful and/or loosened orthopedic implants, including cystic or osteolytic lesions containing diseased, inflamed and/or infected tissues and/or implant wear debris. The method and apparatus of the present invention are also intended to facilitate the preparation of a suitable cavity or bleeding bone bed following removal of diseased tissue, and delivery of biomaterials (both flowable and non-flowable) into the space around the loosened implant and/or within bone tissue so as to enhance bone strength and implant fixation. The primary purpose of the percutaneous placement of these biomaterials into the space around the loosened implant and/or within bone tissue is to alleviate pain and improve functional outcome for medically-compromised patients suffering the debilitating effects of a failed total joint device with local bone loss, without necessitating open revision surgery.

### BACKGROUND OF THE INVENTION

Currently, orthopedic implants are commonly used as a means to treat a variety of defects in bone tissues. Often implants replace joints of the skeletal structure, such as the knee, hip, shoulder or ankle. The bone damage within the joint can arise from multi-factorial issues including trauma, disease, infection, or wear-and-tear (e.g., mechanical wear of the joint bearing surfaces). Osteoarthritis prevails as the most common cause of joint deterioration. When the pain and functional symptoms rise to the level of intolerance by the patient, a surgical option is typically chosen to replace the joint or a portion of the joint. Total Joint Arthroplasty (TJA) has proven to be the treatment of choice to resolve the pain and loss of function from end-stage osteoarthritis. The significant clinical success of total joint arthroplasty procedures over the last several decades has fueled its use in even younger, more active patients, and therefore has driven the need for continuous improvement in implant function and longevity.

Orthopedic implant construction is usually of metal, plastic and/or ceramic materials that must be compatible with, and remain well fixed to, natural bone tissues, while providing biomechanical functionality sufficiently equivalent to the native joint. Despite providing, in most cases, many years of effective function, over time the implants can become loose, causing pain and ultimately requiring revision surgery. Revision surgery is an open invasive procedure during which the loosened components are removed and replaced.

The most common joint replacement procedures are Total Hip Arthroplasty (THA) and Total Knee Arthroplasty (TKA). The prevalence of THA and TKA procedures in the United States is high, with over 1,000,000 procedures performed annually. The high incidence of arthritis, the growing demand for increased mobility and quality of life, and the success of joint replacement surgery over the recent decades has resulted in an estimated 7 million individuals living with artificial hips and knees in the United States. Improved techniques, tools, materials and surface coatings have all contributed to the success of hip and knee implants. In particular, improved understanding of the biocompatibility of materials and their wear characteristics has proven essential to extending the life and performance of the prosthetic implants. Nonetheless, the data reported from national registries suggest revision risks of 5 to 20% ten years following primary THA. This need for revision surgery is typically due to implant loosening.

The long term degradation of implant materials resulting from wear or corrosion typically introduces foreign body particulates within the bone tissue, and consequently leads to a variety of adverse biological reactions, including bone resorption (osteolysis), pseudotumor formation near the implant, painful cysts, and/or systemic reactions.

Adverse biological reactions from particulate debris vary among individuals, perhaps due to genomics or to other factors less well understood. The formation of an abnormal interface tissue between a loosened implant and the surrounding bone or bone cement mantle is a common finding during revision surgery, regardless of the length of implantation time. This abnormal interface tissue results from abnormal biological processes which are initiated in response to the high interface shear stress caused by normal joint loading.

By way of example but not limitation, in the case of a cemented femoral hip prosthesis, loosening starts from de-bonding at the bone cement interface and subsequent inflammation, caused by cement debris and/or polyethylene debris and/or metal wear debris. The resulting chronic inflammatory response eventually produces a pseudomembrane of granulomatous interface tissue including activated macrophages, fibroblasts, giant cells and osteoclasts, similar to the pannus characteristic of arthritic joints. Inflammation also causes local bone resorption adjacent to the implant, weakening the cortical wall of the bone and increasing the risk of periprosthetic fracture. In addition, this granulomatous interface tissue has a very low stiffness which may allow the femoral component implant to rotate and/or subside within the medullary canal of the femur during activities of daily living, which in turn causes pain. Similar loosening is observed with the acetabular component of the THA as well, which may cause displacement, dislocation and/or migration within the pelvis with significant bone loss.

During typical open revision surgery, the loosened implant is removed along with the fibrous tissue and/or fragments of bone cement to allow for the preparation of a new bone bed to accept a larger implant.

Occasionally, osteolytic cysts will form in the periprosthetic bone, presumably due to the presence of wear debris as well as the abnormal or changed stress/strain environment within the bone. These osteolytic cysts may be progressive and unrecognized, and therefore may present a risk to the patient of implant loosening and/or bone fracture. When recognized radiographically, bone cysts must be monitored carefully, because the bone cysts may trigger the need for a surgical intervention.

Revision surgery typically involves the removal of failed implants and their replacement with new implants in a single surgery and, in the case of massive bone loss, may also require bone grafting with structural bone grafts. In the case of bone cysts or bone erosion without structural compromise, surgical curettage and bone grafting with autologous or synthetic biomaterials may be utilized to encourage long term healing and to strengthen compromised bone.

Revision surgery is the only procedure which has proven efficacious in the treatment of loosened total joint prostheses. However, revision surgery is very expensive and has a high morbidity and mortality rate, particularly in elderly patients (who are the majority of revision patients). In patients with cardiac insufficiency, revision surgery often has major complications such as myocardial failure and/or coronary artery disease. Many patients are not eligible for revision surgery because the risk of mortality is considered to be too high. There is no alternative treatment for such patients, who are then typically wheelchair-bound for the remainder of their life. The clinical need for a less traumatic alternative to revision surgery for treatment of loosened prostheses is therefore clear.

Often the loosening phenomena of joint prostheses is categorized as septic or aseptic. If septic, the loosening of the prosthesis can only be resolved with aggressive antibiotic treatments and often explanting of the prosthesis and waiting for the infection to resolve before replacement of the prosthesis. In the case of aseptic loosening, the course of treatment is typically watchful waiting until the pain becomes too great, and then conducting revision surgery to resolve the aseptic loosening. These revision surgeries come at a high cost (monetary, surgery risks, reduced success of surgical outcomes, etc.) and it is generally preferential to postpone or avoid revision surgery if possible. However, if the condition of the prosthesis deteriorates, it generally requires surgical intervention if the patient can tolerate full surgery under general anesthesia.

It is important to note that the terms “septic” and “aseptic” are commonly used in the medical field to identify the cause of loosening of a prosthesis (or other orthopedic implant), however, there are other intermediary conditions and contributing factors which are not well understood and which can also be the cause of loosening of a prosthesis (or other orthopedic implant). Thus, there is some indefiniteness when characterizing the loosening of a prosthesis (or other orthopedic implant) as “septic” or “aseptic”. It should be appreciated that the intent of the present invention is to address the conditions of pain and functioning associated with a loosened implant and not the underlying cause of the loosening.

At present there are several experimental approaches to address the problem of aseptic loosening. One is a preventative approach, where bisphosphonate compounds, especially alendronate, are used to minimize aseptic loosening. The bisphosphonate compounds may be used as either a systemic medication or as a component of the cement used to secure the prostheses (see U.S. Pat. No. 5,972,913, and International (PCT) Patent Publication No. WO 96/39107). However, although bisphosphonates are known to produce an increase in skeletal bone density, they have not been shown to have a significant effect in treating periprosthetic osteolysis itself. Thus it remains to be seen whether bisphosphonates have a useful role to play in the prevention of aseptic loosening.

A therapeutic approach to the problem of aseptic loosening was proposed by de Poorter et al. using gene therapy in order to destroy the granulomatous interface tissue and subsequently stabilize the prosthesis. The proposed method of de Poorter et al. is performed in three steps: injection of a viral vector; injection of a prodrug aimed at killing the granulomatous interface tissue; and rinsing the osteolytic cavities with saline and refixation of the hip prosthesis with percutaneous bone cement injection under radiological guidance (see FIG. 1). Initial human clinical studies demonstrated improvement in walking distance, patient independence and pain relief in most patients. However, the proposed method of de Poorter et al. had a number of medical complications that precluded successful clinical implementation, and was prohibitively expensive, including a significant seven day in-patient treatment regimen in a hospital to monitor drug therapy.

The percutaneous drug delivery and cement procedure of de Poorter et al. demonstrated efficacy in some patients, and de Poorter et al. suggested two important principles to ensure success of the procedure, including:

(1) fibrous interface tissue must be removed to provide space for the in-flowing cement and to optimize the cement-bone interface and the cement-implant interface; and

(2) the cement flow must be “contained” proximally and distally within the bone cavity in order to prevent the cement from escaping into soft tissues.

Implants which are well fixed to bone with bone cement tend to be pain free, and the de Poorter research seems to suggest re-cementing in a minimally invasive fashion is a reasonable alternative in select patients. However, the method of de Poorter et al. has not demonstrated an effective means to remove the fibrous interface tissue and contain the flow of cement. Furthermore, the proposed method of de Poorter et al. utilizes conventional bone cement (e.g., polymethyl methacrylate, also known as PMMA). Such bone cement can migrate from a deployment site before it sets and the polymerization of such bone cement is an exothermic process that can cause tissue necrosis.

In younger or more active patients, it is clinically desirable to refix implants or repair bone with biological materials or bone grafts that encourage natural healing and long term bone remodeling. Fixation may be also be achieved by biological fixation after the diseased tissue has been removed, provided that the biological fixation material can be delivered to the periprosthetic space. Even in the absence of symptoms of aseptic loosening, such as pain, bone pathology suggesting pending implant failure is often discovered during routine imaging studies, including bone cysts or erosion, osteolysis, or fibrous tissue at the interface between bone and prosthesis. In these cases, treatment with injectable biological or synthetic material may be indicated to avoid catastrophic failure. Therefore, the need for percutaneous, less-invasive surgical options for bone repair remains essential in orthopedic surgery.

### SUMMARY OF THE INVENTION

Accordingly, the present invention comprises the provision and use of a novel method and apparatus that provide a minimal, or less-invasive, surgical means for treating aseptically loosened implants (and/or septically loosened implants and/or implants that have become loosened by circumstances other than septic or aseptic conditions) without the need to remove other well-functioning components which are fixed to bone.

The present invention comprises a novel approach for the refixation of a loosened implant to bone, wherein the novel approach comprises:

providing access to a boundary region between the implant and the bone;

removing abnormal interface tissue, wear debris and/or bone cement debris from the boundary region; and

inserting bone fixation material into the boundary region so that the bone fixation material engages the implant and the bone and thereby effects refixation of the loosened implant to the bone.

In one preferred form of the invention, there is provided a method for the refixation of a loosened implant to bone, the method comprising:

providing access to a boundary region between the implant and the bone;

removing abnormal interface tissue, wear debris and/or bone cement debris from the boundary region; and

inserting a thermoplastic polymer into the boundary region so that the thermoplastic polymer engages the implant and the bone and thereby effects refixation of the loosened implant to the bone.

In another preferred form of the invention, there is provided a method for the refixation of a loosened implant to bone, the method comprising:

providing access to a boundary region between the implant and the bone;

advancing a tool into the boundary region and using that tool to mechanically loosen abnormal interface tissue, wear debris and/or bone cement debris within the boundary region;

removing the loosened abnormal interface tissue, wear debris and/or bone cement debris from the boundary region; and

inserting bone fixation material into the boundary region so that the bone fixation material engages the implant and the bone and thereby effects refixation of the loosened implant to the bone.

In another preferred form of the invention, there is provided a method for the refixation of a loosened implant to bone, the method comprising:

providing access to a boundary region between the implant and the bone;

removing abnormal interface tissue, wear debris and/or bone cement debris from the boundary region;

inserting a balloon into the boundary region; and

inflating the balloon so that the inflated balloon engages the implant and the bone and thereby effects refixation of the loosened implant to the bone.

In another preferred form of the invention, there is provided a method for the refixation of a loosened implant to bone, the method comprising:

providing access to a boundary region between the implant and the bone;

removing abnormal interface tissue, wear debris and/or bone cement debris from the boundary region; and

inserting an elastomeric polymer body into the boundary region so that the elastomeric polymer body engages the implant and the bone and thereby effects refixation of the loosened implant to the bone.

In another preferred form of the invention, there is provided a method for the refixation of a loosened implant to bone, the method comprising:

advancing a balloon cannula into a boundary region between the implant and the bone;

inflating the balloon of the balloon cannula so that the balloon engages surrounding bone, stabilizing the balloon relative to the bone and sealing the perimeter of the cannula to the surrounding bone; and using the balloon cannula, inserting bone fixation material into the boundary region so that the bone fixation material engages the implant and the bone and thereby effects refixation of the loosened implant to the bone.

In another preferred form of the invention, there is provided a balloon cannula for accessing a boundary region between an implant and bone, the balloon cannula comprising:

a shaft having a distal end and a proximal end;

a balloon mounted to the shaft adjacent the distal end of the shaft;

a first lumen formed in the shaft and in fluid communication with the interior of the balloon; and

a second lumen formed in the shaft and opening on the distal end of the shaft.

In another preferred form of the invention, there is provided a system for accessing a boundary region between an implant and bone, the system comprising:

a drill guide block for mounting against a surface of tissue overlying the bone; and

a cannula configured for attachment to the drill guide block.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a prior art approach for treating aseptic loosening of a prosthetic implant;

FIG. 2 is a schematic view showing one preferred methodology for identifying an aseptically loosened implant;

FIGS. 3-8 are schematic views showing a novel method for refixation of prosthetic implants to bone tissue;

FIGS. 9-12 are schematic views showing the stages of implant loosening and refixation with the novel method of FIGS. 3-8;

FIGS. 13 and 14 are schematic views of a novel balloon cannula for use in the refixation of prosthetic implants to bone tissue;

FIGS. 15-17 are schematic views showing a novel steerable balloon cannula for use in the refixation of prosthetic implants to bone tissue;

FIGS. 18-24 are schematic views showing an exemplary refixation procedure for the refixation of prosthetic implants to bone tissue;

FIGS. 25 and 26 are schematic views showing a balloon cannula being secured to the side wall of a bone;

FIGS. 27 and 28 are schematic views showing a stabilizing drill guide which may be used to stabilize a cannula during the refixation of prosthetic implants to bone tissue;

FIGS. 29 and 30 are schematic views showing a multi-component cannula which may be used with the stabilizing drill guide of FIGS. 27 and 28;

FIGS. 31 and 32 are schematic views showing a device which may be used for debridement/emulsification/curettage of abnormal interface tissue during the refixation of prosthetic implants to bone tissue;

FIGS. 33-35 are schematic views showing a novel method and apparatus for total ankle fixation;

FIGS. 36-38 are schematic views showing another novel approach for refixation of prosthetic implants to bone tissue;

FIGS. 39-41 are schematic views showing still another novel approach for refixation of prosthetic implants to bone tissue; and

FIGS. 42-44 are schematic views showing yet another novel approach for refixation of prosthetic implants to bone tissue.

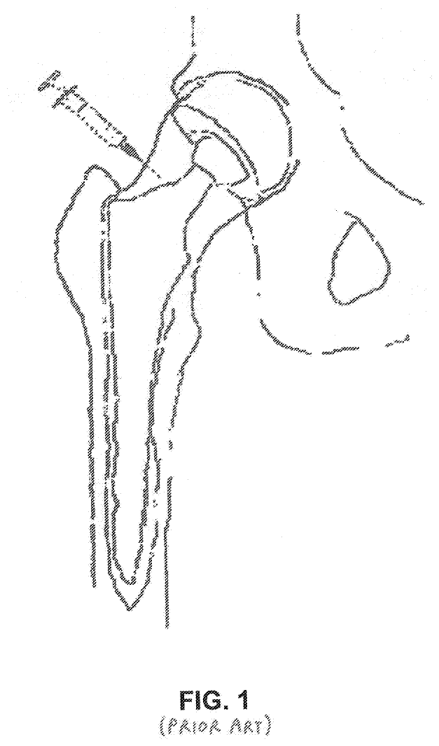

### DETAILED DESCRIPTION OF THE PREFERRED EMBODIMENTS

### The Novel Refixation Procedure in General

The present invention comprises the provision and use of a novel method and apparatus that provides a minimally invasive means for treating an aseptically loosened implant (and/or a septically loosened implant and/or an implant that has become loosened by circumstances other than septic or aseptic conditions) without the need to remove other well-functioning components which are fixed to bone.

For clarity, the present invention will hereinafter generally be discussed in the context of treating an aseptically loosened implant, however, it will also be appreciated that the present invention may also be used to treat a septically loosened implant (in which case additional steps will typically be taken to treat the site of infection) and to treat an implant that has become loosened by circumstances other than septic or aseptic conditions (in which case additional steps may be taken, depending on the cause of loosening).

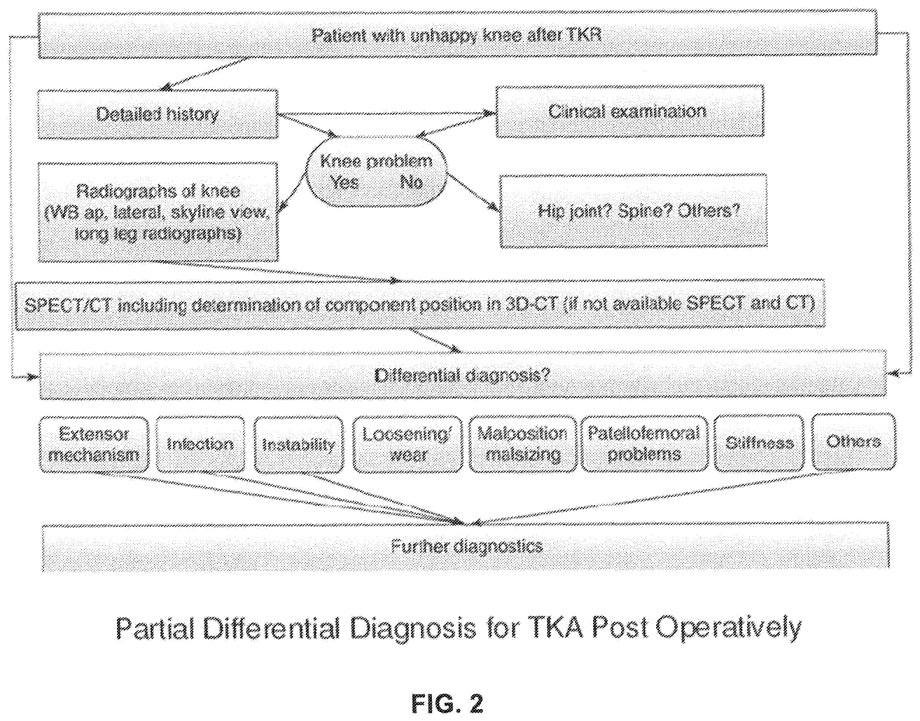

The first step is to identify the loosened implant as aseptic through testing and imaging. The surgeon performs a differential diagnosis using a variety of modalities. See FIG. 2.

In an exemplary example, the invention comprises the evaluation and differential diagnosis of the loosened implant and a determination of the root cause of the osteolysis. The tools for analysis use imaging techniques that are known to detect abnormalities in bone structure surrounding the implant. These imaging techniques include, but are not limited to, X-ray, nuclear, PET, MRI and other imaging modalities that may include marker systems. In addition to imaging, the local tissue and blood is sampled in order to differentiate between aseptic loosening and loosening caused by infected tissue. Upon identification that aseptic loosening is the root cause, a novel step-wise treatment using tools, techniques, and materials is implemented to resolve the osteolysis.

Accordingly, the present invention also includes the step of passing a cannula percutaneously (via image guidance if needed) to the site of the bone tissue which is to be treated. This cannula (and associated attachments for flow control) is used to flow treatment fluids into the bone. The flow of treatment fluids is used to perform a lavage of the site so as to remove damaged tissues and extraneous fluids. Additionally, tools may be advanced through the cannula to remove damaged tissues. The cannula is then used to inject bone fixation materials (preferably flowable biomaterials such as polymers and cements) into the space between the implant and the surrounding bone so as to fill in the voids between the implant and the surrounding bone and thereby re-afix the implant to the surrounding bone.

In an embodiment, during delivery of the bone fixation materials (e.g., flowable biomaterials such as polymers or cements), a feedback system using measurement, volume, pressure or visualization, and/or a combination of these means, is used to determine the proper amount of bone fixation material to be deployed so as to produce the desired result. The invention may further include a means for indicating to the practitioner that the prescribed amount of bone fixation material is in place. In addition, other detection feedback loops may be provided for the set-up times and temperatures, or other parameters, that may be required to ensure safety and proper delivery of the bone fixation materials.

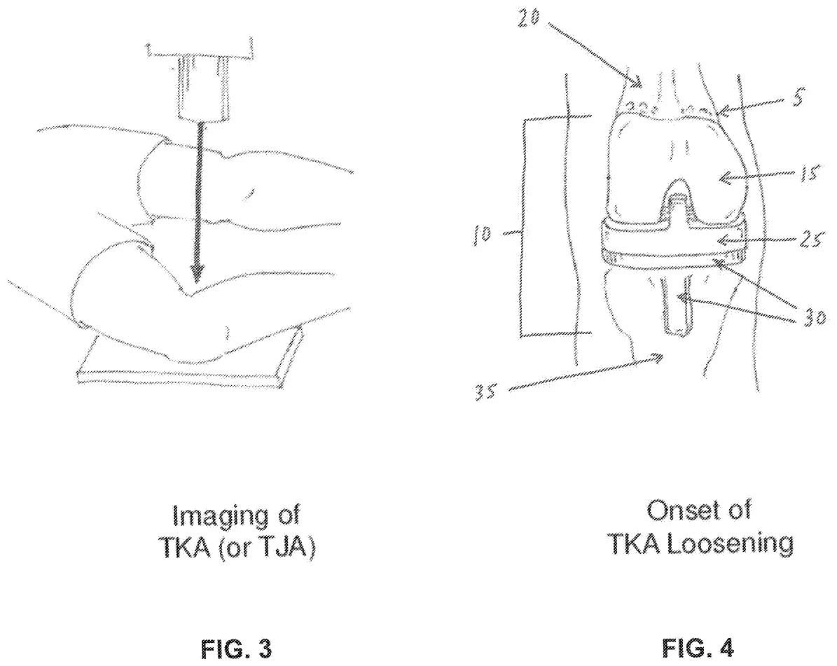

FIG. 3 is a schematic view illustrating the technique of imaging a prosthesis and the surrounding bone structure in order to identify the characteristics of the bone tissue interface that would indicate osteolysis of the bone tissue surrounding the prosthetic implant.

FIG. 4 is a schematic view of a total knee arthroplasty (TKA), where the bone, implant and cement interface region 5 is the location where the bone tissue would typically begin to lyse. The total knee implant 10 has three components: a femoral component 15 which is set into the femur 20, a medial spacer 25, and a tibial component 30 which is set into the tibia 35.

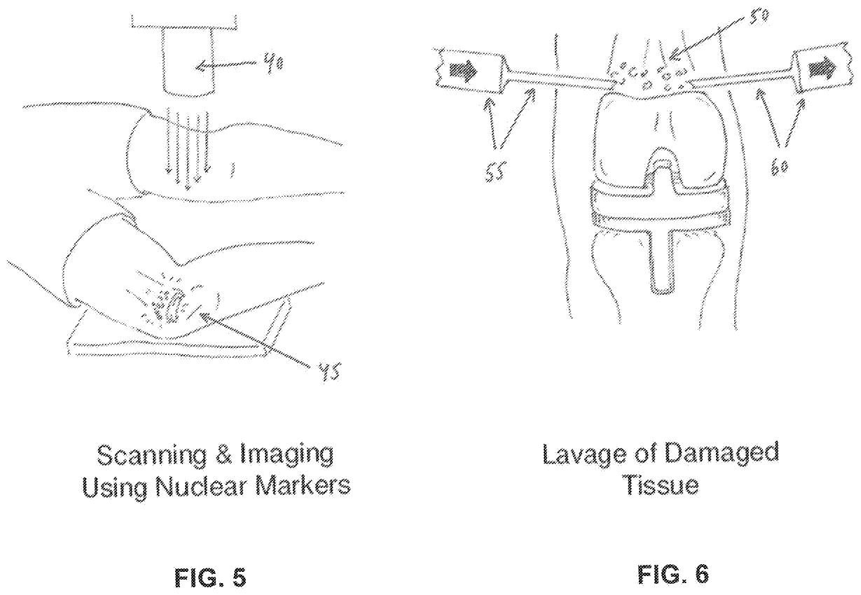

FIG. 5 is a schematic view showing further imaging 40, with or without markers (such as nucleotides) to further differentiate the type of osteolysis in the total knee arthroplasty (TKA) 45, so as to determine that the implant loosening is either septic or aseptic.

FIG. 6 is a schematic view showing the lavage treatment of the aseptic osteolytic bone 50 using percutaneous pressurized flow through an input cannula 55 and an output (discharge) cannula 60.

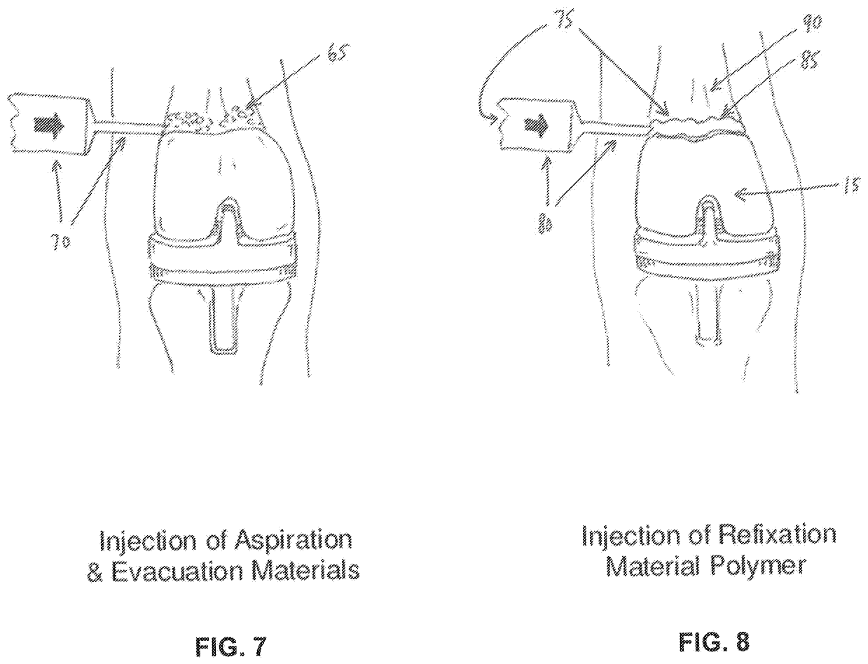

FIG. 7 is a schematic view showing further treatment of the lavaged osteolytic tissue 65, with a secondary injection of preparatory material 70, where preparatory material 70 is adapted to reduce any infection and to improve reception of the bone fixation material (e.g., polymer adhesive) that is delivered in the following step. Evacuation or negative pressure (not shown) may be applied to further assist in the preparation of the site for treatment.

FIG. 8 is a schematic view of the next step, which comprises the percutaneous injection of bone fixation material 75 (e.g., a polymeric material), via a cannula 80, to osteolytic site 85. The bone fixation material fills the bone voids and attaches to (i) the healthy bone tissue 90, and/or (ii) the implant (e.g., femoral component 15), and/or (iii) the original in-place cement which is residual from the initial surgery.

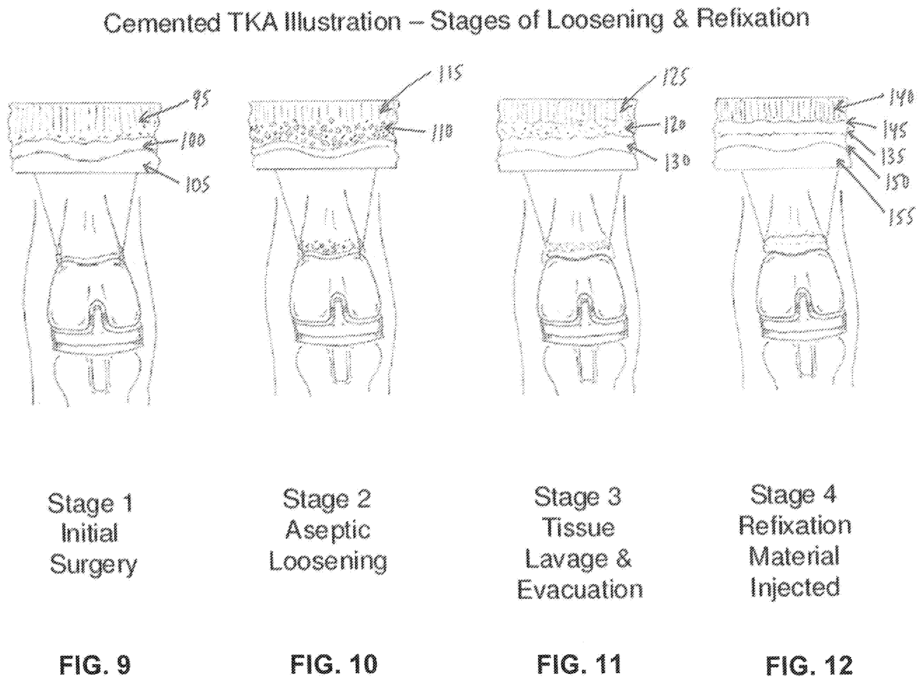

FIGS. 9-12 are a series of schematic views showing the foregoing steps, with the drawings depicting enlarged sections: in FIG. 9, the layers of materials (i.e., viable bone structure 95, bone fixation material 100 (e.g., polymeric cement), and metal implant 105) are depicted in the initial stage of static asymptomatic total knee arthroplasty (TKA); FIG. 10 is a section view showing osteolytic bone 110 at the margins of the implant or cement layer, and at the margins of intact viable bone tissue 115; FIG. 11 shows the osteolytic bone removed at 120 and bordered on one side by healthy bone 125 and either cement or implant material 130 on the other side; and FIG. 12 shows the treated site where a new layer of bone fixation material 135 (e.g., polymeric material) is implanted into the void and adheres to bone 140 at the upper layer 145 and to the existing cement 150 or the prosthetic implant material 155 at the lower layer.

In one form of the invention, the bone fixation material comprises an adhesive polymer, in which case the adhesive polymer binds to the adjacent bone, implant and/or existing bone cement, whereby to stabilize the implant and minimize future bone lose.

In another form of the invention, the bone fixation material comprises a non-adhesive polymer, in which case the non-adhesive polymer fills the voids between the adjacent bone, implant and/or existing bone cement, whereby to stabilize the implant and minimize future bone loss.

Note that where the bone fixation material comprises an adhesive polymer or a non-adhesive polymer, the polymer may be a thermoplastic polymer which is flowable at one temperature (i.e., a temperature above body temperature) and settable at another temperature (i.e., so that it is solid at body temperature).

In another form of the invention, the bone fixation material comprises conventional bone cement (e.g., PMMA), in which case the bone cement binds to the adjacent bone, implant and/or existing bone cement, whereby to stabilize the implant and minimize future bone loss. Significantly, where the conventional bone cement comprises PMMA, polymerization of the PMMA bone cement can cause tissue necrosis, since the polymerization of PMMA is an exothermic process. However, the heat generated by the polymerization of the PMMA bone cement can sometimes also be helpful, since the heat of PMMA polymerization may help treat an infection.

Still other bone fixation materials will be apparent to those skilled in the art in view of the present disclosure.

By way of example but not limitation, the bone fixation material may comprise:

nonresorbable polymers (e.g., Polyetheretherketone (PEEK), polyurethane, polyethelene, polypropylene, acetal, ultra-high-molecular-weight polyethylene (UHMWPE), and PMMA);

bioresorbable polymers (e.g., polylactides (PLA), polyglycolides, polydioxanone, trimethylene carbonate, polyorthoester, and polycaprolactone (PCL);

elastomers (e.g., silicone, gutta percha, rubber, and thermoplastic elastomers (TPEs)); and

natural materials (e.g., collagen, hydroxyapatite, tricalcium hosphate, bioglass, calcium sulfate, bone morphogenetic proteins (BMPs) and other growth factors).

Note that regardless of the composition of the bone fixation material, the bone fixation material is preferably delivered under pressure so as to ensure effective filling of the voids between the host bone, implant and/or existing bone cement. It is important to fill such voids so as to stabilize the implant and minimize future bone loss.

In accordance with the present invention, there is also provided novel apparatus to facilitate effecting the foregoing steps, optionally with additional steps or sub-steps, for the refixation of an implant to bone.

### Novel Balloon Cannula

One aspect of the present invention includes passing one or more novel cannulas percutaneously, via image guidance, to the sites of the bone tissue which are to be treated, for the purpose of introducing lavage fluids to the treatment site, and/or for introducing probes, cutters, etc. to emulsify and remove diseased tissue and/or foreign debris in the periprosthetic space or cyst, and/or for delivering therapeutic agents to the treatment site, and/or for introducing bone fixation material (e.g., polymer adhesive) to repair the bone or refix the implant to bone.

One embodiment of a novel cannula is a balloon cannula comprising a hollow tube (i.e., a shaft) and a handle capable of releasably locking a co-axial solid metal obturator (with a sharpened tip) to the hollow tube, and a balloon attached to the hollow tube and inflatable to stabilize the cannula in bone. The novel balloon cannula is intended for manual operation to penetrate skin, soft tissue and bone, whereby to gain access to the subcortical (or other desired) regions of bone, for instance, within a bony canal, where the novel balloon cannula can be used in a procedure to refix an implant to surrounding bone. As is well known in the art of the surgical biopsy of bone tissue, access to the subcortical (or other desired) regions of bone is typically facilitated by a small stab incision in the skin with a sharp scalpel. The novel balloon cannula is then inserted into bone tissue in a manner similar to the insertion of a bone biopsy needle into bone tissue. The metal components (e.g., the shaft, the obturator, etc.) of the novel balloon cannula provide radiopacity and, therefore, the novel balloon cannula is readily detectable by fluoroscopic or CT imaging, and may be placed at any angle or depth in accordance with clinical need.

The novel balloon cannula has its gripping handle joined to the proximal end of the hollow tube (e.g., shaft), and the removable, slip-fitting, slightly longer, rigid, co-axial obturator is removably disposed within the hollow tube. The obturator has a sharp metal tip at its distal end and lockable knob at its proximal end. When the obturator is disposed within the hollow tube (i.e., shaft) of the balloon cannula and the two components are thereafter locked together, the balloon cannula and obturator can be advanced as a unit through the skin by hand or with light mallet blows until the sharp tip of the obturator breaks through the cortex of the target bone. Alternatively, if preferred, the rigid obturator may be removed from the hollow tube when the outer surface of the bone is first contacted and then the rigid obturator may be replaced with a power drill or manual drill so as to facilitate cutting into the bone. Once the balloon cannula is in the desired position, its balloon is inflated to secure the balloon cannula in position. If desired, the distal end of the hollow tube (i.e., shaft) of the balloon cannula may include roughened surfaces or threads designed to tap into the bone cortex in order to maintain the position and axis of the balloon cannula. Alternatively, an external clamp or jig may be configured to securely hold the balloon cannula in the desired position. Such clamp or jig may be attached to a movable lockable arm which, on its other end, fixes to a rigid structure such as the side rail of a surgical table. This configuration may be preferred when more than one balloon cannula is used for the novel treatment, or when bone quality is not sufficient to hold the balloon cannula in position.

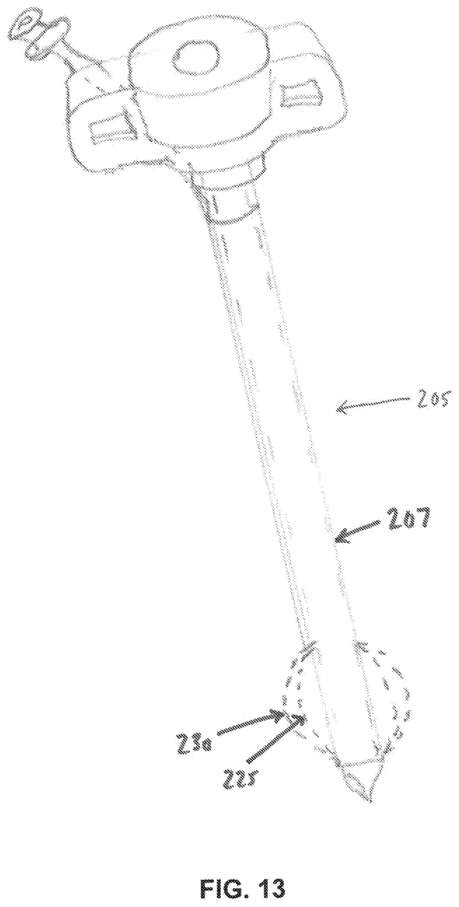

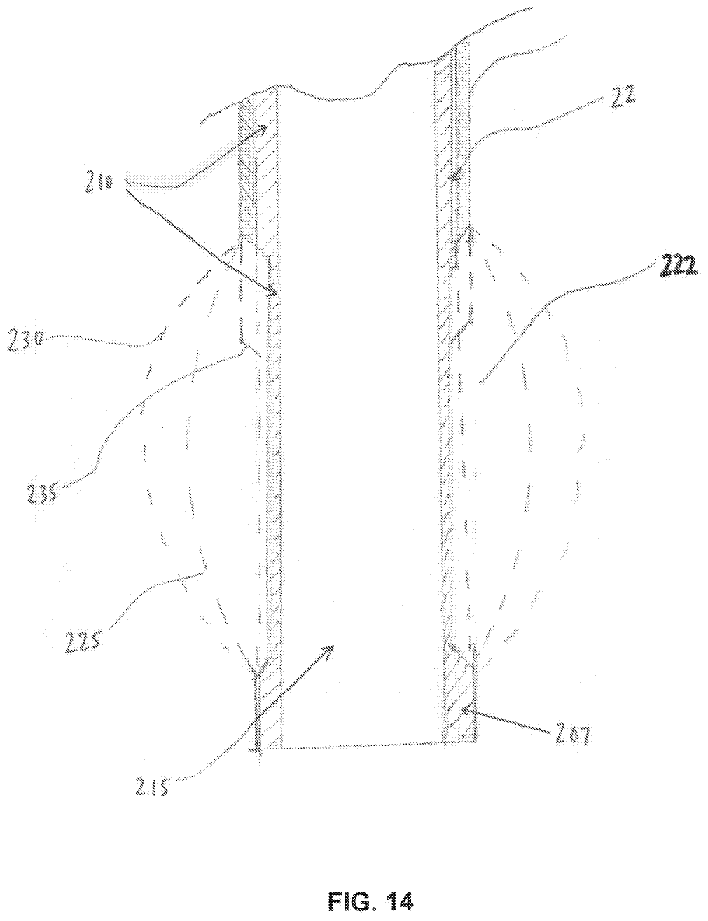

In one form of the invention, and looking now at FIGS. 13 and 14, there is provided a novel balloon cannula 205 which comprises a shaft 207 having a tapered (or reduced diameter) distal end 210, and having at least two lumens, a delivery lumen 215 forming the center lumen of shaft 207 and configured to deliver fluids, tools, therapeutic agents and bone fixation material (e.g., polymer adhesive) to the therapeutic zone, and an inflation/deflation lumen 220 with an inner diameter preferably matching the outer diameter of the reduced distal end 210. Inflation/deflation lumen 220 is in fluid communication with the interior 222 of an expandable balloon 225, such that when fluid is introduced into inflation/deflation lumen 220 under pressure, balloon 225 expands (as seen at 230) at the distal end of the cannula. Fluid within balloon 225 can also be removed via inflation/deflation lumen 220, whereby to deflate balloon 225.

To facilitate insertion of balloon cannula 205 through the bone cortex and into the bony channel (or other desired bone region), balloon 225 is initially provided in a collapsed, folded state disposed within tapered distal end 210 of shaft 207, and held within a retractable polymer or metal sheath 235 which is coaxial with shaft 207. Sheath 235 may be retracted manually by the operator prior to balloon deployment, or it may self-retract by engagement with the bone cortex during the insertion of the cannula (having a diameter that is sufficiently larger than the drilled bone hole) so as to preclude placement of the sheath within the bone canal.

### Steerable Balloon Cannula

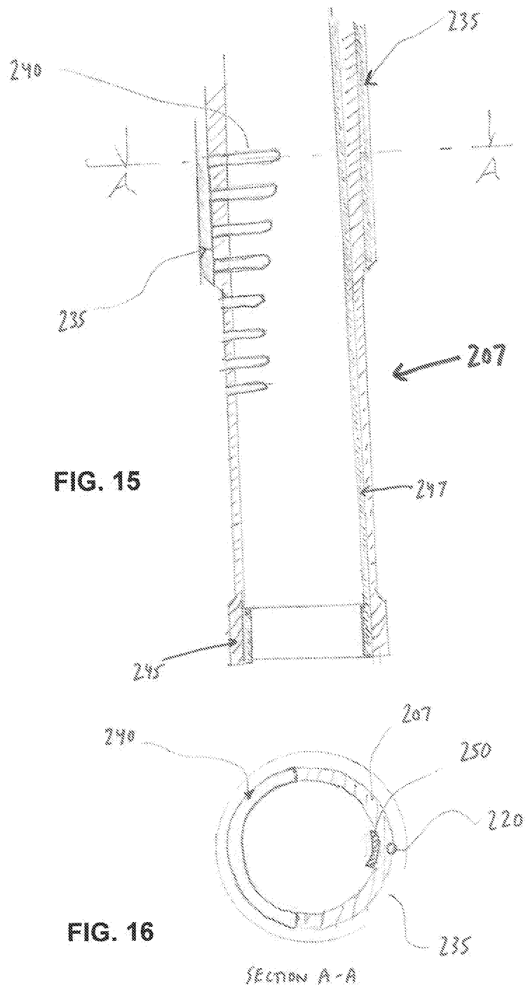

In one embodiment, balloon cannula 205 may be flexible and steerable at its distal end so as to facilitate targeting a location within the bone after placement in the cavity (see FIGS. 15-17). The distal end of balloon cannula 205 may incorporate slots 240 in the end 245 of shaft 207 so as to reduce its stiffness when sheath 235 is retracted. An inner tube section 247 is slidably disposed within shaft 207. Inner tube section 247 may have its distalmost portion welded to the distal end of flexible shaft 207. Inner tube section 247 is laser cut in a configuration which forms an effecter wire or band 250 which is capable of transferring a tension load to the distal end of the flexible cannula (so as to steer the distal end of the flexible cannula) when inner tube section 247 is moved proximally relative to flexible shaft 207.

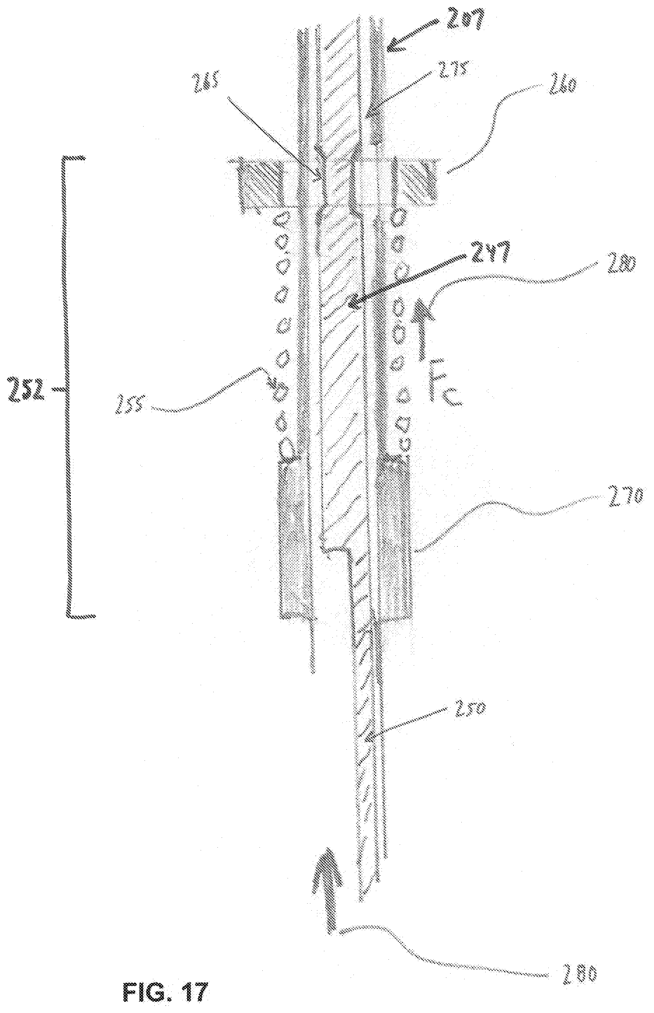

Exemplary steering design features for a balloon cannula are shown in FIG. 17, which shows a washer clip-spring-shoulder assembly 252 formed flexible shaft 207 proximal to slots 240 of shaft 207. Washer clip-spring-shoulder assembly 252 comprises a compression spring 255 sandwiched between a washer clip 260 and a solid shoulder 270. Washer clip 260 is snap-fit to an inner tube section 247 at a cutaway slot 265 of inner tube section 247. As shown in FIG. 15, inner tube section 247 slidably fits within shaft 207, and as shown in FIG. 17, shaft 207 comprises a clearance slot 275 for receiving washer clip 260 so as to allow constrained axial movement of inner tube section 247 relative to flexible shaft 207.

Inner tube section 247 has its band or effecter wire 250 integrated along its length distally to the end of shaft 207, where it is attached (e.g., via welding) to the distal end 245 of shaft 207. Since shaft 207 is flexible distally, a proximal force 280 on the band or effector wire 250 also deflects the distal end of shaft 207. At the same time, washer clip 260 transfers the force of compression spring 255 to inner tube section 247. Washer clip 260 is within axial slot 265 within shaft 207 proximally, and therefore its position may be altered within the handle assembly by a depressable button or rotatable knob (not shown) which alters the position of compression spring 255, and therefore alters the force on effecter wire or band 250, resulting in deflection of the tip of shaft 207 to a different bend radius.

The inner diameter of the steerable balloon cannula may accommodate a trocar (not shown) with a sharp, rigid tip to temporarily straighten and stiffen the distal end of the steerable balloon cannula to impact the tip into the bone, fibrous layer or bone cement which is adjacent to the distal end of the steerable balloon cannula. Targeting may be further enhanced by the use of an endoscope placed through one of the cannulas at a location within the joint capsule (not shown), as is well known in the art of hip arthroscopy. Subsequent inflation of the expandable balloon 225 compresses damaged tissue, forming a cavity in the tissue while also stabilizing the balloon cannula, so as to allow fluid communication from the filling end of the balloon cannula (outside of the patient's body) to the bone cavity within the bone.

Once the desired cannula portals have been established, the bone cavity may be accessed with saline lavage, or chemonnucleolytic agents of the sort known in the art, such as gellified ethanol, chymopapain, ozone formulations, specialized synthetic or naturally occurring enzymes, or other agents designed to dissolve or obliterate abnormal fibrous tissue without having a deleterious effect on healthy bone. The cannula portals may also be used to advance tools to the therapy site in order to remove abnormal fibrous tissue. Alternatively, it may be desirable to inject a bioactive drug or drug carrier (such as chitosan hydrogel), or flowable biomaterial filler (such as demineralized bone matrix, calcium phosphate cement, morselized autologous bone chips, etc.) so as to enhance healing. Then the lumen of the balloon cannula may be used to flow bone fixation material (e.g., adhesive polymer) into the interior of the bone so as to re-secure the loose prosthesis.

### Exemplary Refixation Procedure

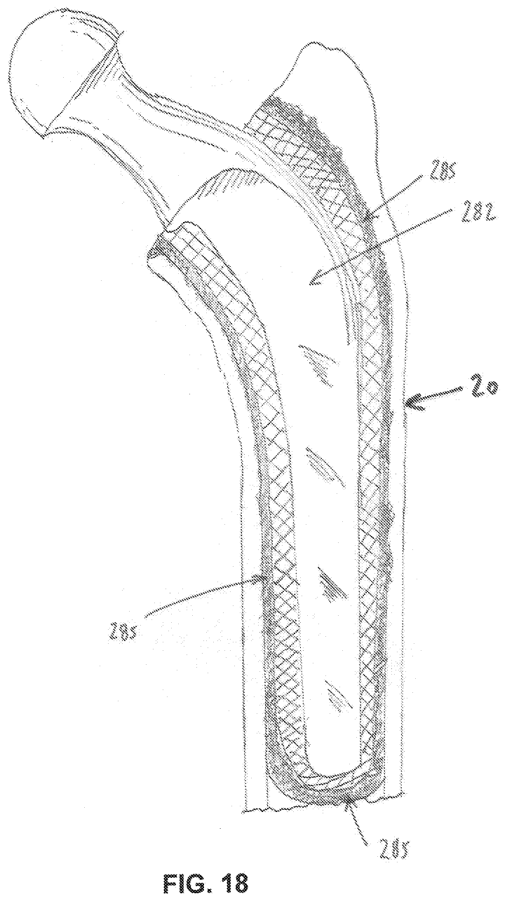

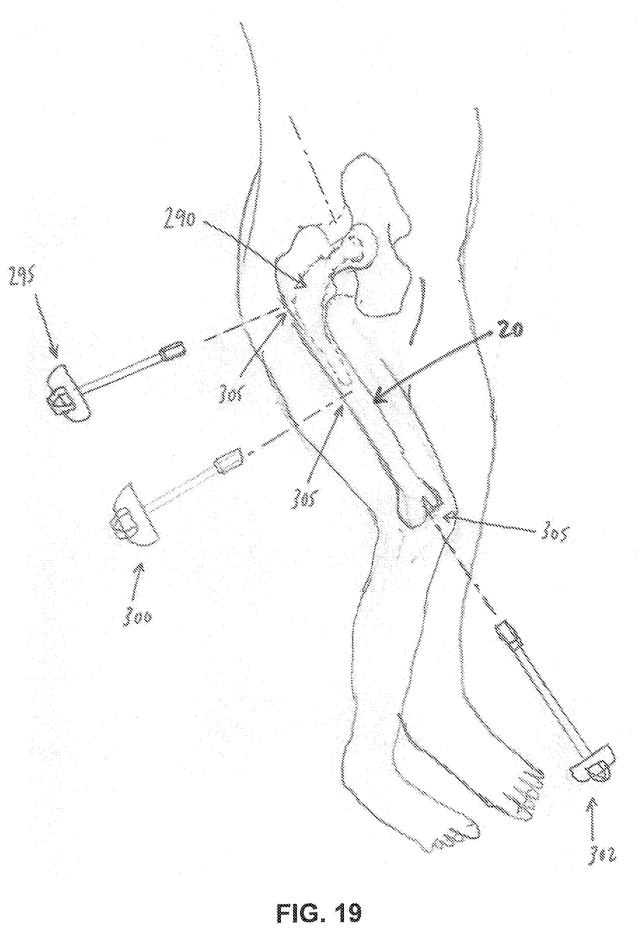

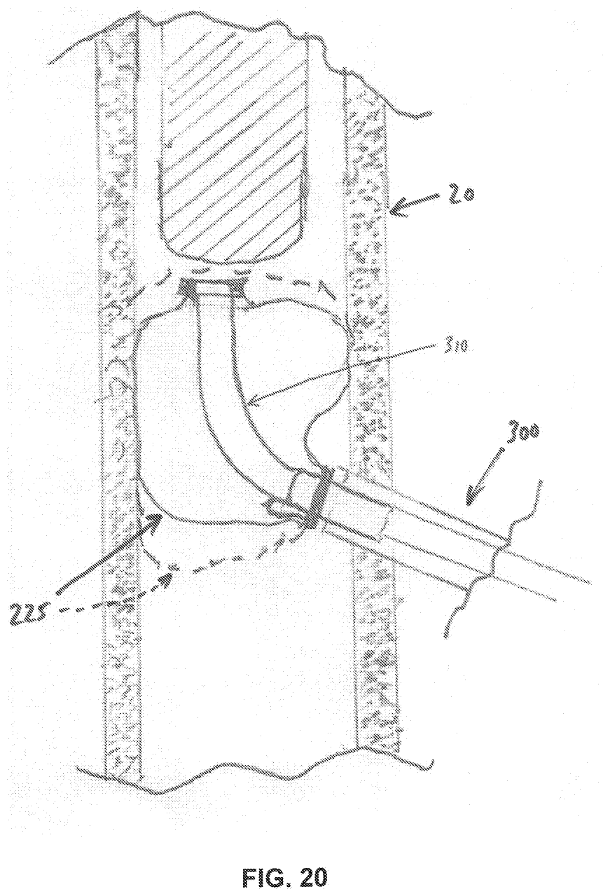

A representation of a loosened total femoral cemented hip implant 282, having a fibrous tissue interface 285 with bone, is shown in FIG. 18. A proposed surgical method will now be discussed using the foregoing embodiments as applied to total hip refixation. Three balloon cannulas with distal expanding tips are affixed to the femur of a human patient as shown in FIG. 19, wherein the femur has an implant 290 which has loosened and/or exhibits periprosthetic tissue pathology. Two balloon cannulas are shown penetrating the lateral aspect of the femur, one balloon cannula 295 being located proximally and one balloon cannula 300 being located distally near the mid-shaft of the femur. Another balloon cannula 302 may be delivered to the distal aspect of the femur using a portal 305. The balloon of the cannula near the distal aspect of the loosening femoral stem (i.e., the aforementioned balloon cannula 300) is shown inflated in FIG. 20. A lumen 310, capable of enabling fluid flow under pressure, extends from the proximal handle of the balloon cannula to the distal aspect of the balloon so that fluids may ingress and egress to the periprosthetic region (i.e., the region between the implant and the adjacent bone, which is sometimes referred to herein as “the boundary region”) without flowing distally in the intramedullary canal, due to the blocking seal established by the inflated balloon across the intramedullary canal.

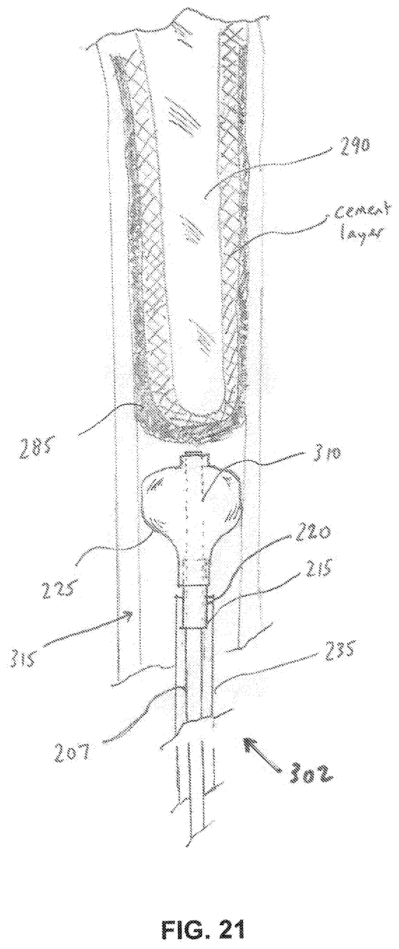

The preference of cannula portal locations is driven by clinical preference dependent upon patient presentation. For instance, a portal 305 (FIG. 19) may be introduced from within or proximate the knee joint and advanced within the intramedullary canal parallel to the axis of the femur. Once placed within the bony canal and advanced to the desired location, the balloon may be inflated to cause the balloon walls to expand within the bone channel and contact the inner wall of the cortex (or other desired bone region). See, for example, FIG. 21, where a balloon cannula (i.e., the aforementioned balloon cannula 302) has been advanced up the intramedullary canal of the femur so as to approach the distal end of the prosthesis, and then the balloon has been inflated so as to seat against the inner wall of the cortex 315. By inflation of the balloon, the distal end of the balloon cannula is thereby temporarily secured to the bone, creating a seal in the bone canal, wherein only the delivery lumen 215 of the balloon cannula is in fluid communication with the bone cavity near the distal end of the prosthesis, as shown in FIG. 21. Note that in addition to creating a seal to contain material introduced through a balloon cannula, the inflated balloon can also be used to compress tissue and clear space within the bone before advancing material into the boundary region (i.e., the region between the implant and surrounding bone).

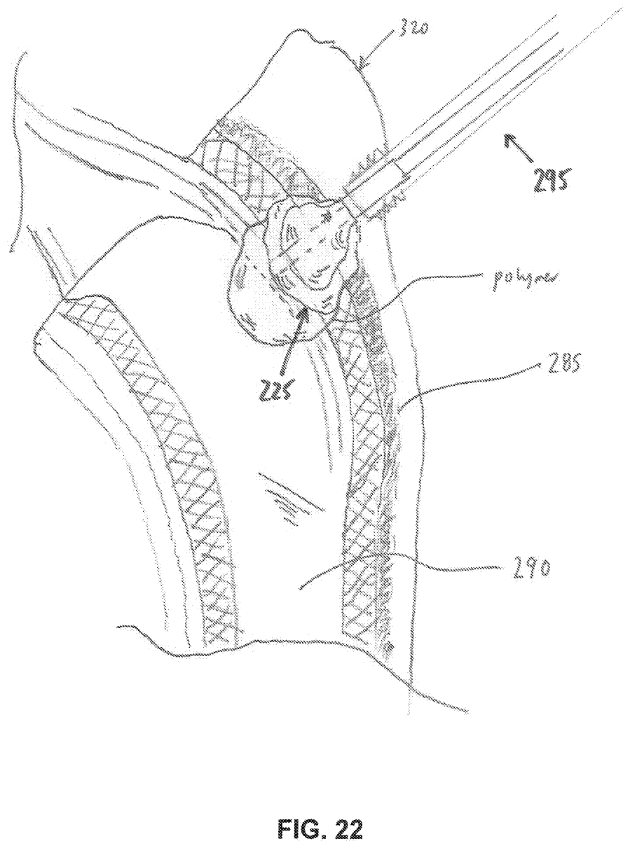

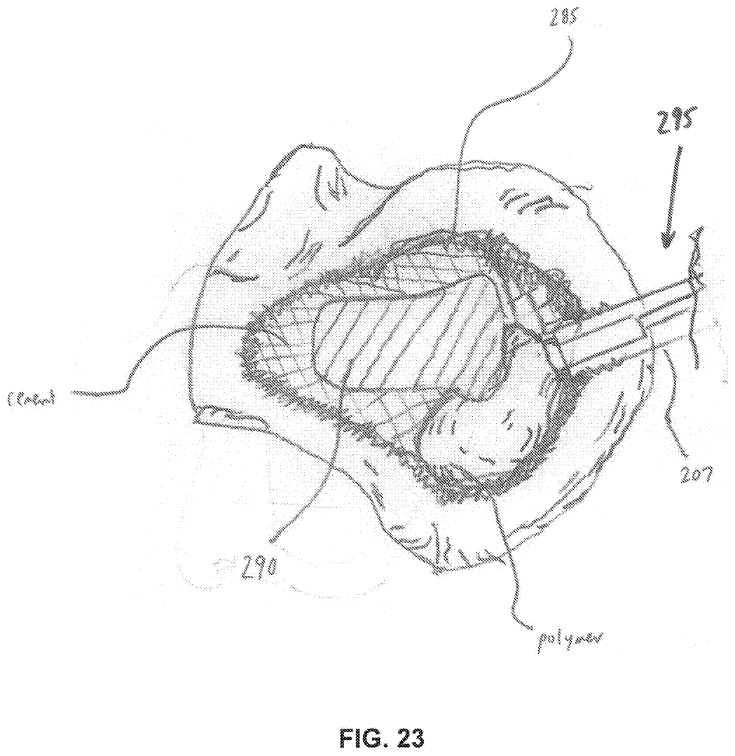

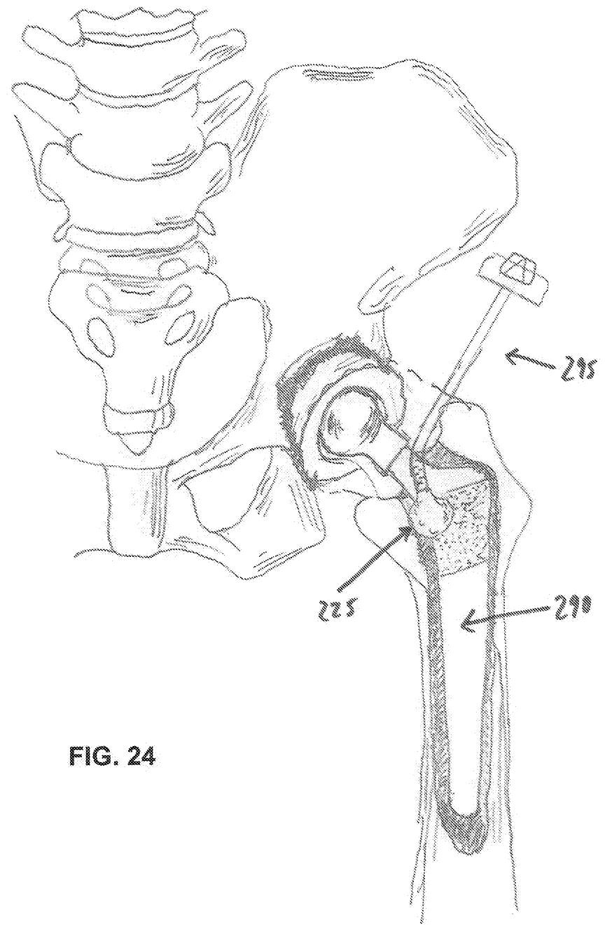

The proposed methods also include conducting imaging studies using arthrography (biocompatible radio-opaque die injection), or using real-time endoscopic visualization, of the fibrous membrane (at the interface of the bone, bone cement or metal implant) to establish the ideal position for additional portals (e.g., so as to adequately address the specific regions which are causing the implant instability). For instance, as shown in FIGS. 22-24, the balloon cannula may be directed into the femur to a position adjacent the implant by advancing through capsular tissue or through the greater trochanter 320, at a desired location and to desired depths prior to balloon inflation. Once all of the desired balloon cannulas have been placed in the patient and their balloons inflated, fluids or therapeutic substances may be injected through the cannula portals, or alternatively, pathological fluids may be evacuated from the space under vacuum. The balloon cannulas may also facilitate the introduction of surgical tools, such as curettes or probes, into the periprosthetic space in order to remove fibrous membrane, weakened bone cement, etc.

Since there is typically more than one cannula portal, one or more of the established cannula portals may be used to suction lavage fluid and dissolved fibrous particles, implant wear debris, or bone cement, etc. from the space between the implant and the bone, until abnormal materials are substantially removed or substantially reduced from the space between the implant and the bone. The use of a second cannula portal (which is effectively sealed by its own inflated balloon) allows for suction of fluid aspirate. The cannula portals also allow for the flushing and removal of radiopaque fluid media used to verify the location and volume of the voids surrounding the implant.

Subsequent to cavity preparation and fluid lavage, the cannula portals provide a means to fill the cavities with bone fixation material (e.g., an adhesive polymer, a non-adhesive polymer, conventional bone cement (PMMA) or another suitable material) so as to stabilize the loose implant in the bone. The bone fixation material is preferably a flowable media and may include bone graft or bone graft substitute. After the refixation procedure is completed, the balloons may be deflated and the balloon cannulas removed.

### Securing the Balloon Cannula to the Side Wall of the Bone

The balloon locking feature at the distal end of the cannula (or the threaded locking feature at the distal end of the cannula) may, alternatively, be used to secure the distal end of the cannula to a single cortical wall of a bone (or other structure) after the cannula tip has been placed in the desired position within the bone or joint.

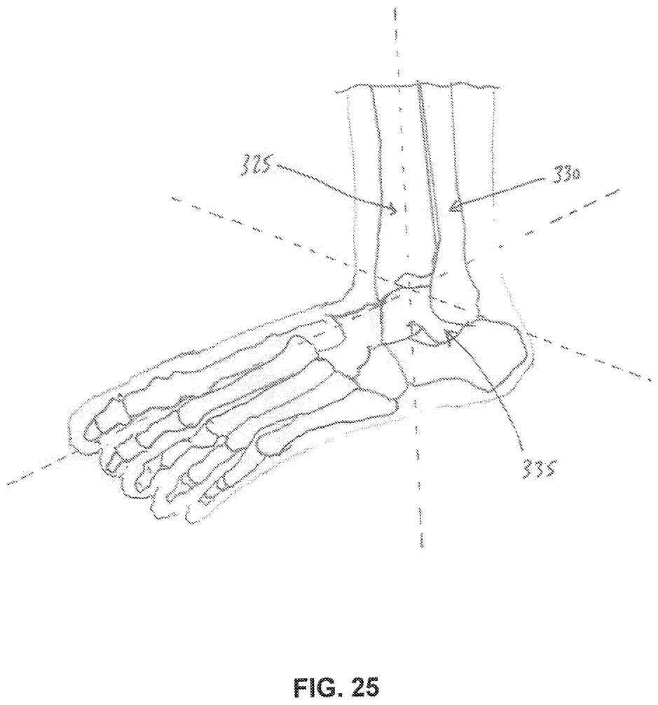

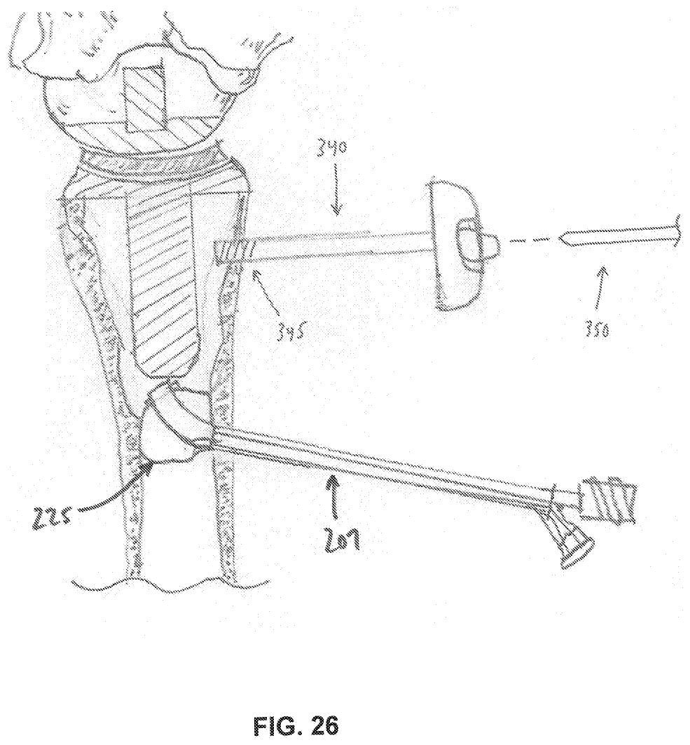

An exemplary application is the ankle joint (FIG. 25) comprising the distal tibia 325, the distal fibula 330, and the talus 335 of the foot. In this form of the invention, and looking now at FIG. 26, the cannula 340, with distally-oriented threads 345, may first be placed over a K-wire or pin 350 that has been previously drilled through the bone at the desired trajectory and to the desired depth near the fixation stem of a tibial implant, as established through pre-operative analysis of bony and soft tissue landmarks. In difficult anatomy, the targeting of the K-wire or pin may be enhanced by the use of interoperative imaging such as CT or fluoroscopic guidance, or by the use of custom drill guide instruments which are pre-fabricated based on image data. In this form of the invention, threads 345 secure the distal end of the cannula to the cortex of the bone, whereupon lavage of the site may be initiated (to be followed by deployment of bone fixation material). Alternatively, the balloon at the distal end of a balloon cannula may be used to secure the cannula to cortical bone (or other structure).

### Stabilizing Drill Guide

Depending on bone quality, and on other anatomical limitations that may preclude secure placement of a cannula portal in bone (e.g., via a stabilizing balloon or stabilizing screw threads), a stabilizing drill guide block may alternatively be employed.

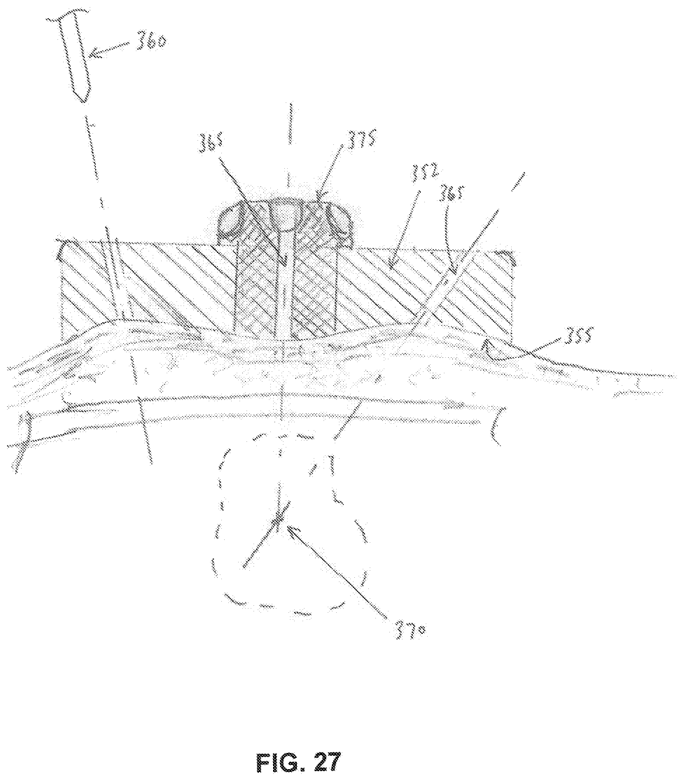

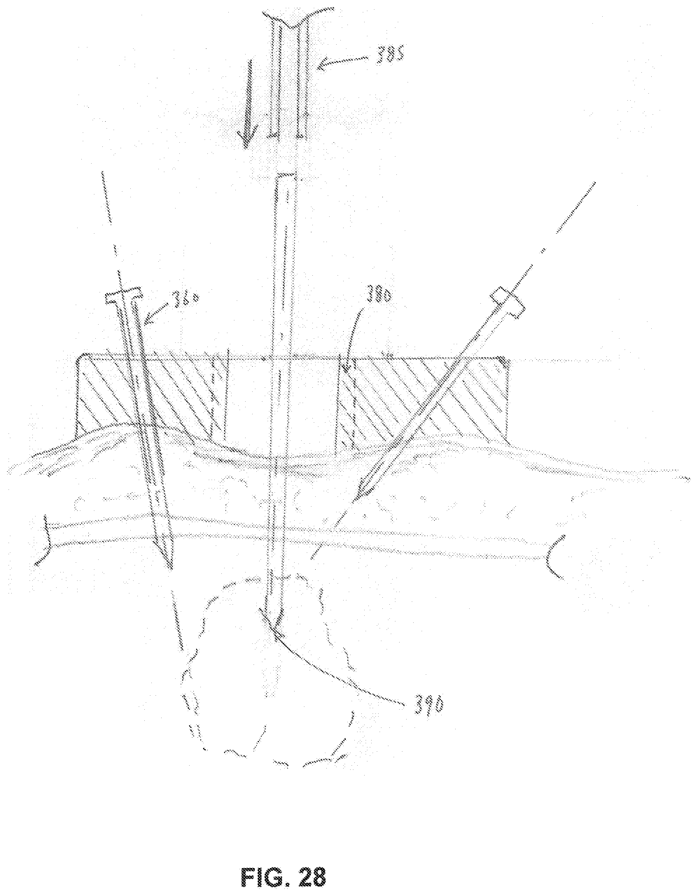

An embodiment of an exemplary drill guide block 352 that may be used to position a K-wire (and, thereafter, a cannula) is shown in FIGS. 27 and 28, as applied to the skin of the patient near the intersection of the distal tibia and the talas bone of the ankle joint. Drill guide block 352 is preferably fabricated in a biocompatible, sterilizable plastic using 3D printing technology so that the contour of the patient-contacting surface 355 of the drill guide block matches the patient so that it may be directly applied to the skin of the patient and secured in place with one or more pins 360. The drill guide block has guide holes 365 designed to target pin placement in bone while avoiding structures at risk (such as nerves or blood vessels). At least one of the pin holes 365 is targeted to the treatment area 370, which is confirmed in real-time with fluoroscopy. Once pins 360 are placed through the drill guide block and into the bone, a pre-designed, generally removable or detachable cylindrical coaxial section 375 of drill guide block 352 may be removed so as to expose a larger area on the patient's skin (i.e., an area large enough to accommodate the cross-section of a balloon cannula, see below). Alternatively, when the drill guide block is fabricated by 3D printing, the geometry of the removal section 375 may be designed with asymmetric or otherwise unique geometries, so that the section is only removable along a selected axis. Note that removable section 375 may include a guide hole 365 and, if a pin 360 is disposed in that guide hole, section 375 is removed along the axis of the pin. When assembled to the block, the geometry 380 (FIG. 28) of the drill guide block and the removable section 375 are designed to resist rotation or distal migration of the removable section 375.

Another variation of the drill guide block includes portal cylindrical guide holes which are oversized to the pin diameter, enabling the use of a removable slip-fit guide sleeve 385 on the pin itself which fits within a guide hole 365. The guide sleeve rigidly secures the pin to the block, but can be removed when a larger diameter drill or cannula is placed along the same path.

The exemplary skin-contacting drill guide block is ideally suited for well-defined anatomy where the skin is close to the target bone without significant interpositional soft tissue, e.g., a bone prominence such as the malleolus of the ankle. The drill guide block may be fabricated as a single, relatively small block or it may be fabricated as a multi-part, joinable block so that it may be assembled on the patient with tape or other locking features in order to hold the patient's joint or bone securely in position to accommodate multiple targets and cannula portals. Essentially, the drill block design may allow the operator to position the joint, and maintain stability of the joint, during the placement of the pins and, thereafter, during placement of a balloon cannula.

Any of the guide holes 365 may be designed in accordance with pre-operative image analysis to converge with an adjacent guide hole at a point 390 distal to the housing, as shown in FIG. 28. In these instances, a second pin 360 may be removed and replaced with a rigid endoscopic camera tube, which provides stabilization of the drill guide block as well as providing visualization of the anatomy of interest, such as a cyst wall or a void near an implant. Second or third ports (provided through guide holes 365 or other openings into the interior site) may be utilized for a suction apparatus, as is well established in the art.

Depending on the strength of the bone and the diameter of a desired access port, a trephine or cannulated drill (not shown) may be first slid over a guide wire after the removable section 375 of the drill guide block has been removed from the drill guide block, and then the trephine or cannulated drill may be advanced to the bone surface, whereupon it is rotated manually, or with power, to cut and enlarge the bone portal diameter. If a guide sleeve 385 is used, the trephine drill may be placed into the guide hole 365 after the guide sleeve 385 and pin 360 have been removed. The trephine or cannulated drill has features to allow the operator to measure and note the depth of the penetration into the bone. The inner diameter of the distal end of the trephine or cannulated drill may exceed the diameter of the guide pin so that a plug of bone is removed along with the pin. The opening left after removal of this bone plug is aligned with the opening left in the drill guide block after section 375 has been removed. A balloon cannula may then be advanced through the opening left in the drill guide block after section 375 has been removed, and then advanced through the bone, so as to provide access to an internal site, with the balloon cannula being supported by the drill guide block.

### Novel Multi-Component Cannula

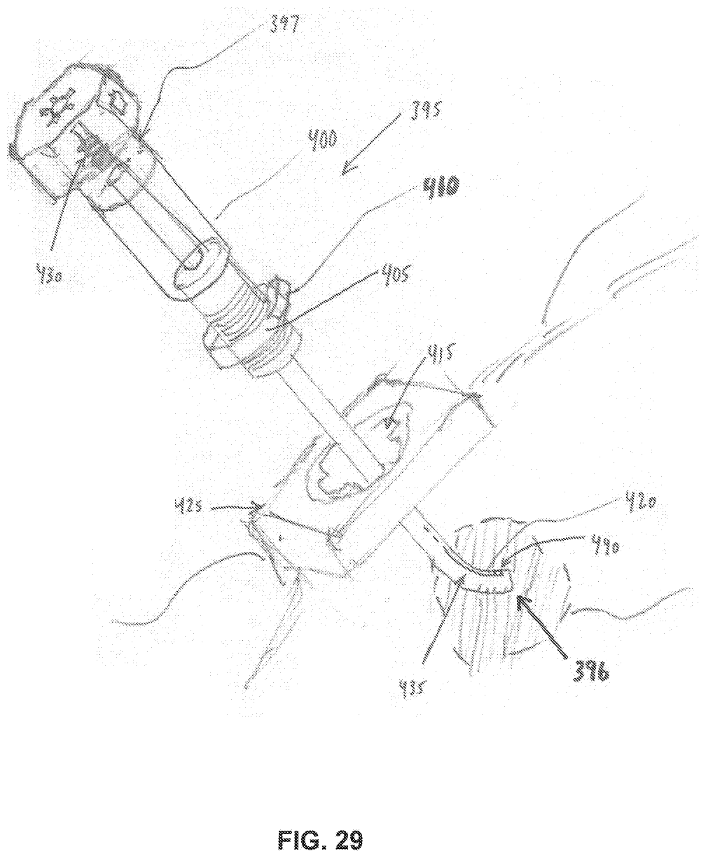

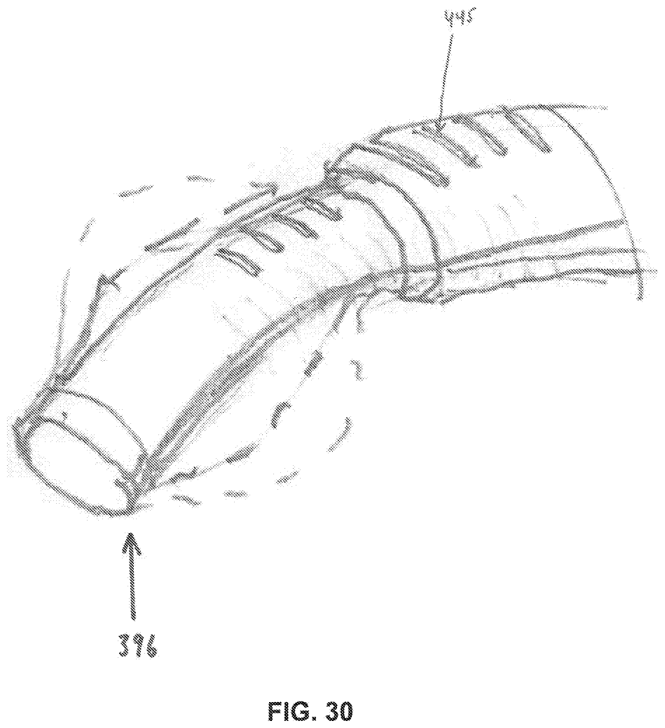

The drill guide block may be used with a novel multi-component cannula 395 which is inserted into the drill guide block as shown in FIGS. 29 and 30. The multi-component cannula 395 has a steerable deflecting tip 396 using a spring-loaded effecter wire or band as disclosed previously herein, and a length-adjustable body component, which is attachable to the custom drill guide block. The length of the cannula is adjustable to match the hole depth measurement provided by the drill or trephine tool, or by quantitative image analysis. The proximal end of the multi-component cannula 395 has a handle assembly 397 housing an adjustable, spring-loaded mechanism to deliver force to an effecter wire or band attached to the distal tip of the cannula, whereby to steer the distal tip of the cannula.

The handle of the cannula shown in FIG. 29 comprises an insert-molded plastic tube 400 with an adjustable locking shoulder 405 which may be set at a length to ensure that the cannula is placed at the proper depth. The locking shoulder 405 of the cannula further comprises a lock button or retractable secondary flanged locking tab 410 which engages a plurality of recesses 415 in drill guide block 352 for the purpose of locking the cannula at the ideal depth and rotational alignment. A rotational and axial lock of the cannula assembly to the drill guide block facilitates use of the steering features 420 located at the distal end of the cannula. The steering features are located within the cannula body.

Exemplary steering design features are those disclosed previously in FIG. 17, comprising a compression spring engaged to a washer clip that is snap-fit on an inner tubular ring which slidable fits within the outer cannula. Alternatively, other steering features may be also used.

In a preferred form of the invention, the steerable cannula apparatus shown in FIG. 29 uses steering features of the sort disclosed in FIG. 17, and includes an inner tube subassembly comprising a rigid proximal aspect and a 10 mm-50 mm long, flexible, steerable cannula tip aspect extending distally from the rotational and axial locking body 425. The tube subassembly includes a compressible, force-producing spring 430 oriented on the axis of the tube assembly which is attached proximally to an effecter wire or band 435. The spring 430, under compression, delivers a tension force to the effecter wire or band 435 where it is bonded to the tip of the outer tube 440. The outer tube is slotted at 445 (see FIG. 30) to reduce its bending stiffness, whereby to allow deflection upon the introduction of the tension force. The proximal spring assembly also includes a means to alter the compression force of the spring to, in turn, change the deflection force and, therefore, the radius of the deflected distal tube.

Since the guide block components comprise radiolucent plastic, the operator may assess the cannula tip position and deflection using routine interoperative imaging.

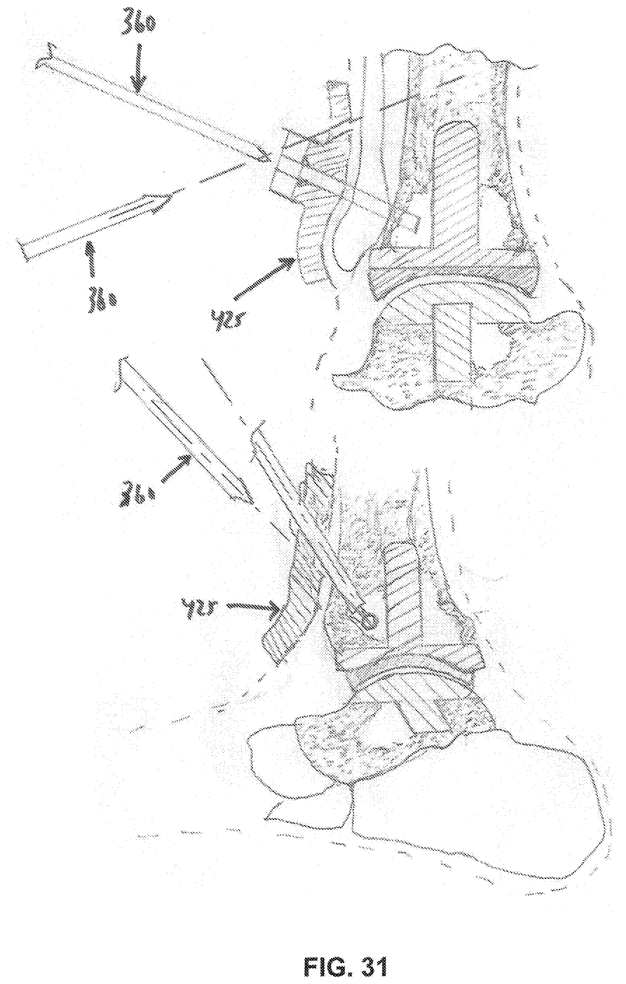

A representation of multiple pins placed through custom guide blocks is shown in FIG. 31.

### Debridement/Emulsification/Curettage

Another aspect of the present invention includes devices and methods for physical removal of the damaged or diseased tissue (e.g., the granulomatous interface tissue) surrounding the implant.

It is well established in the prior art that saline or other fluid may be enhanced by a pressure stream so that the fluid is capable of damaging, cutting or obliterating tissue. Systems are known which employ a nozzle or handpiece having a small diameter orifice to generate a jet stream in response to high pressure fluid. A variety of liquid jet instruments with the capability of delivering a variable pressure stream of liquid for surgery have been developed, including instruments described in U.S. Pat. Nos. 5,944,686, 6,375,635, 6,511,493, 6,451,017, 7,122,017, 6,960,182, U.S. Patent Application Publication No. US2003-0125660, U.S. Patent Application Publication No. US2002-0176788, U.S. Patent Application Publication No. US2004-0228736, U.S. Patent Application Publication No. US2004-0243157, U.S. Patent Application Publication No. US2006-0264808, and U.S. Patent Application Publication No. US2006-0229550, which are all incorporated herein by reference in their entireties.

Prior art configurations of fluid jet instruments do not include the capability of steering them from a location distant from the nozzle so that the specific tissue surrounding a loosened implant may be targeted for cutting within a bony canal. The devices and methods disclosed herein may be deployed through any cannula system, including variations of the aforementioned balloon cannula, the aforementioned threaded cannula, or the aforementioned guide block-stabilized cannula portals and related apparatus disclosed herein, provided that the nozzle and tube assembly are small enough in diameter to be introduced through, and extend beyond, the cannulas. Preferably, the tip is selectively movable or steerable by active or passive mechanisms linking the nozzle tip to a proximal handle, so that the operator can control the direction of the flow at the nozzle from the proximal handle.

It is believed that an active deflector nozzle mechanism would be well suited to a rigid cannula, and a passive deflector nozzle mechanism would be well suited for a cannula with deflecting or steering capability, as disclosed herein.

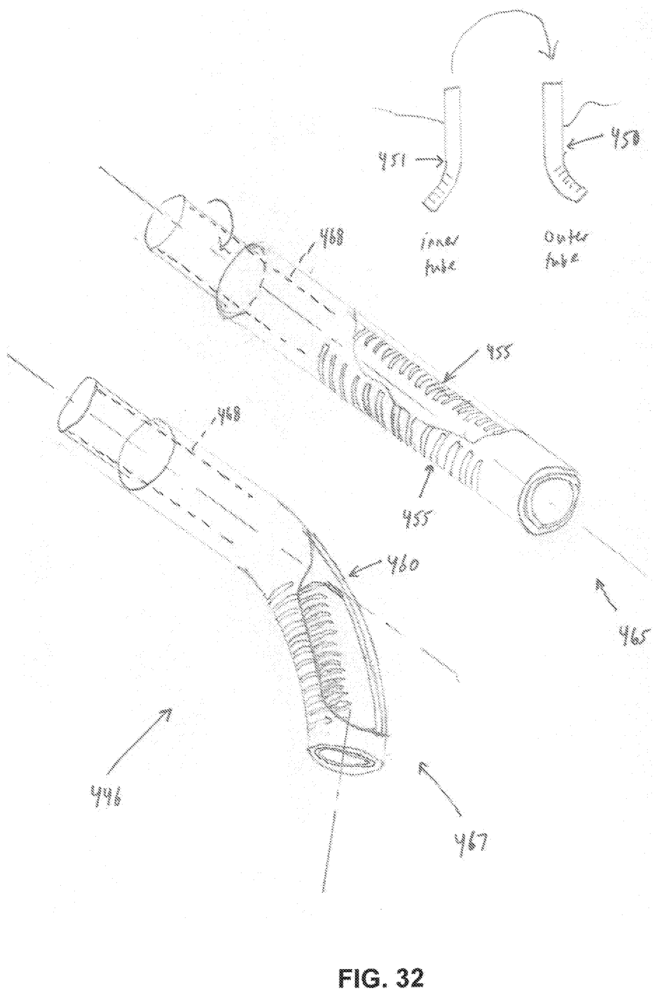

In one exemplary embodiment shown in FIG. 32, there is provided a tip housing 446 wherein the tip housing may be formed of coaxial metal tubes, with the outer tube 450 being formed into an arc with symmetrical laser cut slots to reduce its bending stiffness, and the inner tube 451 having an outer diameter which is sized to fit within the inner diameter of the outer tube 450. Both co-axial tubes have a series of laser cut slots 455 design to reduce the flexural stiffness of both tubes. Preferentially, both tubes or a portion of both tubes may be made of Nitinol or other superelastic material so that the ends of the tubes can be heat-set with the same arc 460, while also having sufficient elasticity to flex into a straight configuration without permanent deformation upon the introduction of an appropriate force. When the tubes 450, 451 are assembled, the arc of the assembly will vary dependent up the axial orientation of the slots, since the slots in each tube effectively control the overall bending stiffness of the tube assembly. For example, as shown in FIG. 32, the tubes 450, 451 are assembled with opposing curves (slots) such that when fully engaged, the tube assembly assumes a straight configuration 465, since the lateral bending force of the outer tube 450 is counteracted by the lateral bending force of the inner tube 451. When the inner tube 451 of FIG. 32 is rotated with respect to the outer tube 450, the tube assembly can be reconfigured into a curved configuration 467. Thus, continuous rotation of the inner tube 451 relative to outer tube 450 will cause the tip of the tube assembly to alternate between straight and curved. The tube assembly could include a manual lever or dial at the proximal, handle end of the device to cause the rotation of inner tube 451, which it turn deflects the nozzle end of the tube assembly.

The embodiment further includes a coaxial fluid conduit tube 468, preferably constructed of reinforced, flexible plastic, contained within the flexible coaxial metal tube assembly that provides resistance to radial expansion force of high pressure fluid in inner tube 451. The internal conduit tube 468 could also be constructed of a small diameter metal tube, with sufficient flexibility to allow the nozzle to flex, or the internal conduit 468 could be manufactured with transverse microslots which are sufficiently small to prevent the outflow of water radially, but effectively reduce the bending stiffness of internal conduit 468.

Another variation of the above embodiment adds design features to the fluid jet flow tube assembly to enable continuous back and forth deflection of the nozzle tip while pressurized with fluid. In this embodiment, the inner slotted metal tube 451 which is sandwiched between the outer tube 450 and the inner fluid conveying tube 468 has turbine or impeller features (not shown) on its proximal end which disrupts the flow of fluid so as to cause the inner tube 451 to rotate within the housing upon the introduction of fluid under pressure within the tubing. The proximal end within the outer tube 450 is formed with one or more slots or blade configurations similar to a turbine so that when under fluid pressure, the inner tube 451 is subject to a rotating force which is proportional to the fluid pressure within the inner tube. These blades may be formed in a Nitinol tube by laser cutting a shaped slot through the tube wall, deflecting the shape inwardly toward the central axis of the tube, and shape setting the tube so as to cause the deflected section to remain in position.

In another variation of this embodiment, the inner tube 451 may be replaced with a solid wire, heat-set with a laterally-deflecting curve at its distalmost aspect, and with a turbine blade housing attached thereto proximally, so that the wire is encourage to rotate upon the introduction of a fluid force. The curved wire at the distal end of the assembly is subjected to the thrust force of the fluid, as well as to the rotating force of the turbine housing, causing the outer tube 450 to alternate between a curved position and a relatively straight position, depending upon the water pressure. The nozzle configuration is housed and bonded at the far distal end of the fluid conduit tube 468, and has one or more small fluid exit ports, for example, on the order of 0.1 mm in diameter, so as to maximize the fluid jet exiting the tube assembly.

During normal use for the above configurations, the introduction of fluid pressure by an externally-powered pump produces a strong fluid jet at the distal end of the tip housing and causes the tip to “wobble”, such that the spray jet trajectory varies in a conical spray pattern, thus increasing contact with materials in the path of the jet, e.g., for cutting or emulsification of tissue.

Alternatively and/or additionally, ultrasonics may be used to enhance emulsification of materials (e.g., bone or other tissue) and/or cutting of materials at the distal end of the assembly.

### Total Ankle Fixation

It should be understood that variations of the novel balloon-stabilized cannula devices and methods described above for removing diseased fibrous tissue can also be used to refix loosened ankle prostheses with bone fixation material (e.g., polymers or bone cement). These devices and methods can be applied to enhance percutaneous or open, primary or revision, total joint replacement surgery in a less invasive fashion. For instance, certain revision total ankle joint replacement procedures are intended to exchange only a worn or damaged interpositional component without necessitating the removal of one or more components which remain well fixed to bone. These open procedures also benefit from devices and methods that are less traumatic and invasive to the patient, and reduce the likelihood of surgical morbidity. Use of less invasive devices and methods may also be employed when access to diseased bones or failed devices is limited by anatomy or scar tissue, especially from previous operations.

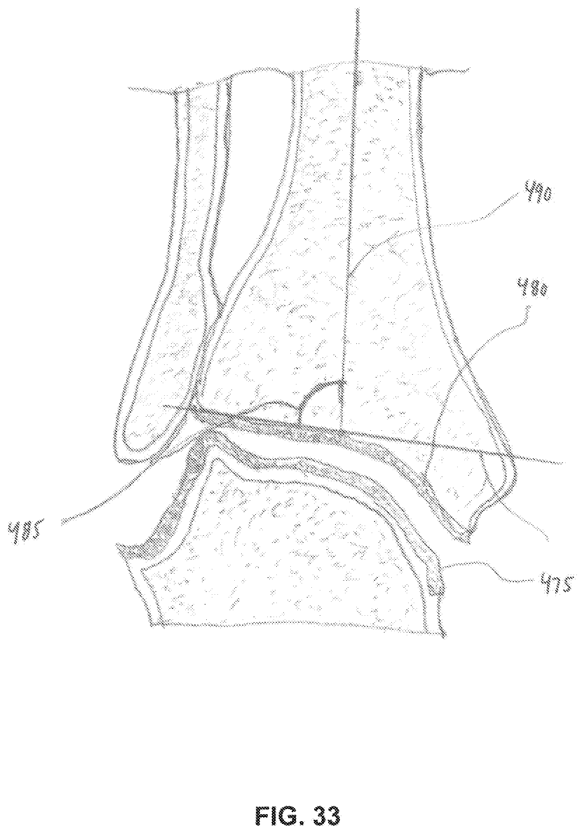

An exemplary embodiment describing less invasive devices and methods for the refixation of a total ankle prosthesis is shown in FIGS. 33-35. In this example, devices and techniques are described relating to the use of a bone fixation material (e.g., a polymer or bone cement) to fix a specially-designed prosthesis to bone in a manner that assures rigid implant fixation while limiting or precluding open or wide surgical exposure, and facilitating placement and pressurization of bone fixation material (e.g., a polymer or bone cement) to the prepared bone interface after both the talar and tibial implants have been positioned in (or on) the prepared bone.

It is well known that viscous but flowable liquid bone cement must form a solid bond between healthy bone stock and the surface of the implant (which may be plastic or metal). Furthermore, once cured, the composite bone-cement-implant interface must have suitable bond strength at both the bone-cement and cement-implant interfaces, which is generally achieved through mechanical interlock and maintenance of a preferred cement layer thickness.

Knowledge and clinical techniques involving the effective use of bone cement has evolved over the years, in particular with hip and knee joint replacement. These techniques include improved methods of bone resection, preparation of surfaces with lavage and drying techniques, use of improved cement formulations, improved implant interfaces, and pressurization to achieve optimal interlocking of cement to the bone and metal implant. These techniques typically involve the injection or placement of cement within the bone prior to implant placement. This technique is difficult (if not impossible) to replicate in smaller, more complex joints such as the ankle or shoulder due to bone exposure limitations, and therefore have not enjoyed widespread acceptance.

Therefore, there remains a need to improve the cementing technique in smaller, more complex and challenging joints.

Total ankle replacement, for instance, has become an acceptable alternative for treatment of end stage ankle arthritis. Clinical results, however, remain inferior to results currently achieved in the knee and hip.

Certain orthopedic principles of total joint replacement of the ankle are described in the prior art for early generations of these devices. Failure of early devices which employed bone cement can be attributed, in part, to inferior cementing technique, as well as to flawed implant designs, high biomechanical loads, and a poor understanding of ankle kinematics.

Aseptic loosening of one or both of the components fixed to bone is the most common cause of early failure of a total ankle replacement. Often, prior to mechanical failure, radiolucent lines and/or osteolytic cyst formation is observed radiographically by the practitioner, perhaps due to excessive wear debris or other, less well understood mechanisms. In such instances, a surgical intervention may be warranted, particular to reinforce compromised bone structures. One potential treatment involves the use of bone cement to reinforce compromised bone and to restabilize the implant.

One aspect of the present invention provides a novel apparatus and method to achieve implant restabilization by proper placement and pressurization of bone fixation material (e.g., a polymer or bone cement) at the loosened bone implant interface. The novel apparatus and method of the present invention provides a means to drill, lavage, and/or remove abnormal fibrous or synovial tissue. The novel apparatus and method provides a novel means to place the bone fixation material (e.g., a polymer or bone cement) within the joint from a minimally invasive path originating from within the bone proximate to the bone-implant interface. The apparatus and method further provide a novel means to inject and pressurize a controlled volume of bone fixation material (e.g., a polymer or bone cement) directed to the bone-implant interface after the implant has been temporarily fixed against the bone surface so as to cause the interdigitation of bone fixation material (e.g., a polymer or bone cement) with both the bone and the implant. The lack of such interdigitation predisposes an implant to early mechanical failure. The apparatus and method further provide a novel means to reinforce the bone-cement-implant composite structure by the use of bone screws placed proximate to the joint prior to the application of bone fixation material (e.g., a polymer or bone cement) and which are subsequently embedded within the flowing bone fixation material. The novel apparatus and method may be used to refix loosened implants (as in the case of an open revision surgical procedure) or to enhance fixation of newly placed implants in a primary open surgical procedure.

Another aspect of the present invention describes devices and methods to apply bone fixation material (e.g., a polymer or bone cement) from within the bone in a minimally invasive fashion to the tibial and talar components which have been positioned and stabilized in the final preferred clinical orientation, but not yet permanently attached to bone. A significant challenge in the application and pressurization of bone fixation material (e.g., a polymer or bone cement) from within the bone after placement of the implant is the maintenance of proper implant alignment within the joint. Loss of alignment during fixation of one or both of the tibial and talar components in this example, for instance, predisposes the implant to early failure. Certain embodiments of the apparatus and method disclosed herein define a means to maintain alignment of the talar and tibia components while injecting bone fixation material (e.g., a polymer or bone cement) with proper pressurization.

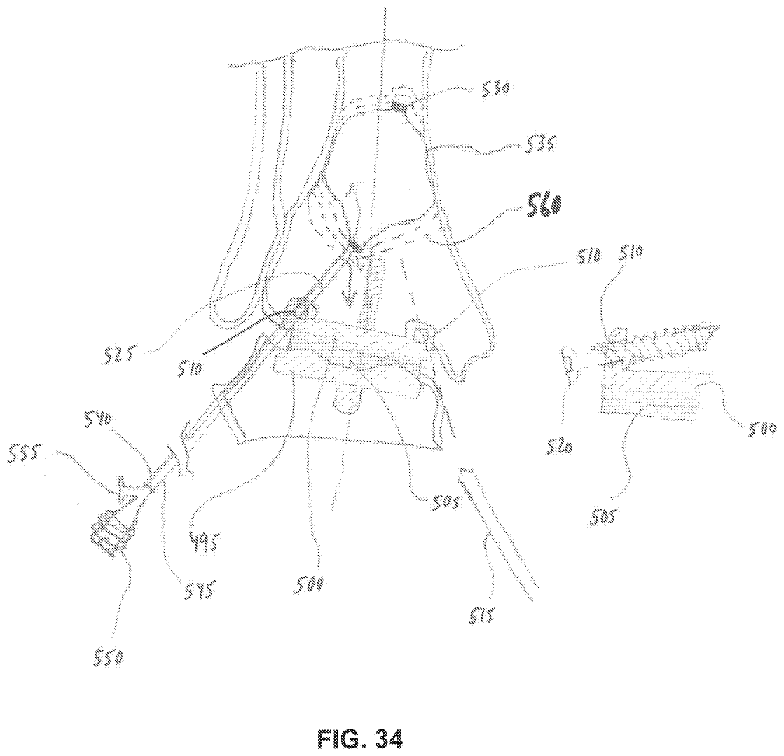

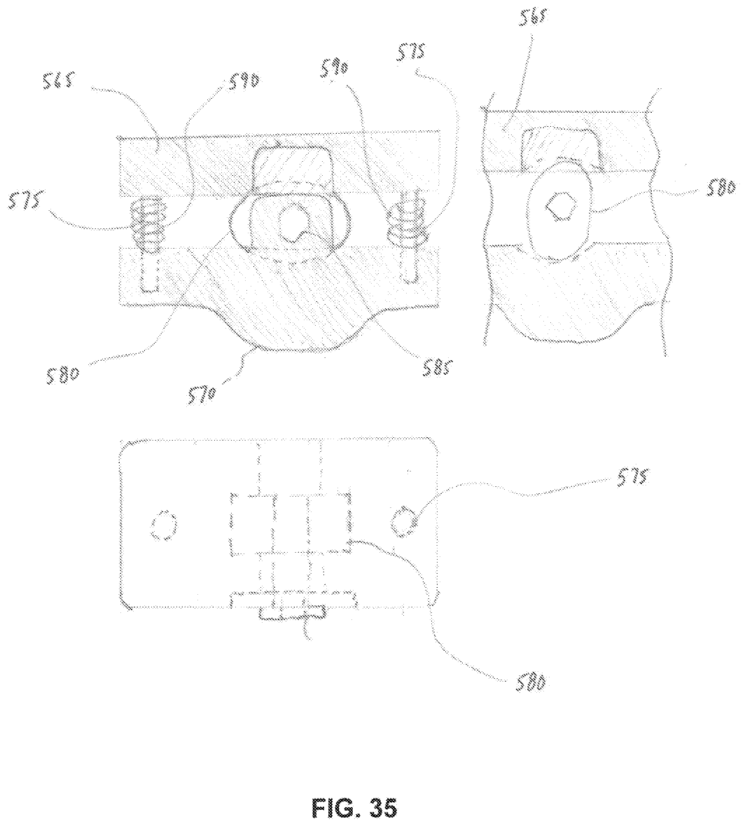

A normal anterior cross-sectional view of the ankle is shown in FIG. 33 wherein the talus articulation 475 is congruent with the tibial articulation 480 in 5 degrees of valgus 485 relative to the anatomical axis 490 of the tibia. In FIG. 34, the talar component 495 and tibial component 500 are shown placed in bone in the anatomically desired 5 degree valgus position, fitting within surgically-resected bone in accordance with bone resection techniques which are well known in the art. As shown, the talar and tibial components are juxtaposed and flush with the bone cut. In this configuration, a temporary spacer 505 is disposed between the talar and tibial components which helps to temporarily align and compress the prosthesis against the flat bone cuts. The temporary spacer 505 has an adjustable height feature as shown in FIG. 35. The force of compression applied to each of the components is dependent upon resultant ligamentotaxis as the stabilizing ligaments and soft tissue of the ankle are stretched in tension.

Additional fixation may be employed, if necessary, on a temporary or permanent basis, by the use of bone screws or pins which may be placed angularly and proximally through anterior-oriented holes 510 in the anterior flange of the prosthesis. The tibial component, for instance, may preferentially have two adjacent holes directed proximally and medially through the strong anterior cortex. In one embodiment, a pilot hole is first drilled into the bone using a drill 515. The pilot hole is drilled into the bone through one or both of the holes 510 in the implant into the softer intramedullary bone canal. A single screw 520 (or Steinman pin, if temporary) may be inserted though one of the holes 510, on a temporary or permanent basis, to further stabilize the prosthesis for subsequent cement injection.

In this preferred embodiment, a novel bone fixation material injector device 525, with an on-board expandable balloon member, is placed through the second anterior portal 510 in the tibial plate device, and extended proximally into the tibial metaphasis to a position proximal to the tibial plate. It will be appreciated that bone fixation material injector device 525 may comprise a balloon cannula 205 discussed above.

The placement of the novel bone fixation material injector device 525 is facilitated by first drilling an access hole through the harder subchondral bone of the distal tibia. The novel bone fixation material injector device 525 is preferentially constructed of a flexible reinforced multilumen polymer tube having at least one radiopaque feature (i.e., radiopaque marker) 530, such as a crimped tantalum or platinum ring, to allow radiographic visualization of the device within the bone, and an expandable balloon member 535 bonded at the distal end. The balloon member is in fluid communication with one of the lumens such that fluid flowing from the proximal end 540 of the tube fills the balloon.

Once positioned, a preferentially radio-opaque fluid is injected into the expandable balloon to cause the expansion of the balloon within the bone canal, preferentially within the metaphasis approximately 10 mm to 30 mm proximally to the subchondral bone. The expanded balloon member contacts the inner cortical walls of the bone, thereby forming a seal against the flow of liquid bone cement.

The second lumen 545 of the novel bone fixation material injector 525 has preferentially a larger diameter than the balloon-filling lumen 540. Both lumens may be bonded to a luer connecter at the proximal end, the larger lumen 545 intended to connect with a bone fixation material-filled syringe 550 or other bone fixation material-filling apparatus, as is well known in the art. The smaller diameter lumen 540, also bonded to a second luer fitting 555 at its proximal end, is intended to connect to a second syringe or filling container having a low viscocity, aqueous media such as saline with an additive to enhance radio-opacity. The smaller lumen 540 is in fluid communication with the balloon 535 at its distal end (the balloon 535 is capable of being folded or collapsed to a minimum diameter for placement into the bone channel), such as is described above. The balloon 535 is preferentially constructed of polyurethane or other compliant materials as known in the art, which is demonstrably capable of expanding within softer bone upon the introduction external fluid pressure. A compliant balloon material continues to expand with increasing pressure, and therefore, in this embodiment, the introduction of sufficient radio-opaque fluid is continued until the bone canal is completely filled by the balloon.

Once the balloon 535 is filled, bone fixation material (e.g., a polymer or bone cement) may be introduced into the larger lumen 545 of the bone fixation material injector device, with the first lumen 545 having sufficient diameter (e.g., 11 gauge or greater) to allow the flow of viscous bone fixation material into the bone space between the inflated balloon and the tibial implant. Since this space between the balloon and the tibial implant is effectively sealed, additional pressure may be delivered by the bone fixation material syringe to encourage the bone fixation material to fill bone spaces and perforations within the subchondral bone which abut the metal interface of the tibial implant device. Specially-designed grooves, channels, holes, or protrusions may also be machined in the surface of the metal implant so that they are enveloped by the bone fixation material with continued pressurization 560.

Once sufficient bone fixation material (e.g., a polymer, bone cement, etc.) has been injected into the sealed space within the bone, as noted radiographically, additional pressure may be optionally employed by increasing the pressure within the balloon seal, which in turn further pressurizes the bone fixation material and maximizes the fill at the interface between the bone and implant, and seals any gaps that may allow the ingress and egress of synovial fluids or wear debris from the joint space.

Alternatively, if needed, both holes in the implant, or a secondary hole along the tibia bone, could be used to employ additional balloon seals, allowing the operator to control the volume of the cavity within the bone proximal to the implant and, therefore, the amount of bone fixation material injected.

An embodiment of the temporary spacer 505 is shown in FIG. 35, wherein the spacer is capable of expanding to a greater height by the use of an internal cam mechanism (or other mechanical or hydraulic means). This feature allows additional pressure to be applied directly to the tibial and talar implants after the bone fixation material (e.g., a polymer or bone cement) has been injected, and further helps to measure the size needed for the articulating plastic bearing component once the bone fixation material (e.g., a polymer or bone cement) has cured. The temporary spacer 505 may be provided to accommodate a variety of varus/valgus or plantar/flexion orientations to facilitate anatomical correction.

The temporary spacer 505 has a tibial surface aspect 565 abutting the implant surface and movable or expandable along the vertical axis relative to the talar aspect 570 via telescoping pins 575 or other means to guide the expansion. The device shown has a cam shaft 580 with a hex drive 585 so that the user can rotate the cam shaft, whereby to cause the tibial and talar surfaces to separate. The cam shaft may include lockable or indexable features (not shown) to allow multiple height adjustments. A spring 590 may be included to ensure that a minimum compression force is maintained against the implant and curing bone fixation material (e.g., a polymer or bone cement) when the cam is disengaged.

### Additional Embodiments of the Present Invention

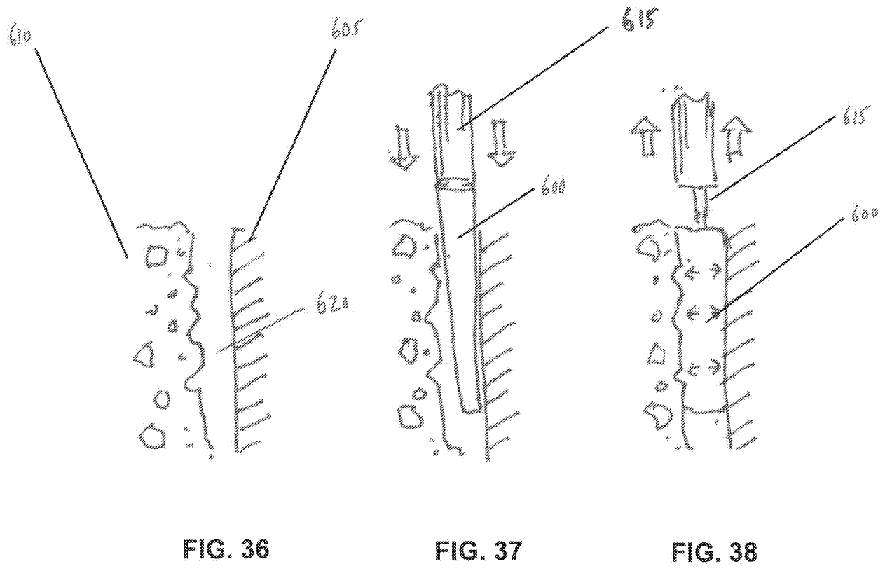

In another form of the invention, and looking now at FIGS. 36-38, expandable biocompatible elastomeric plugs 600 can be inserted or deployed as a filler or as a wedge so as to create an interference fit between a loosened implant 605 and bone 610, whereby to effect refixation of the implant to bone. In particular, a preferred embodiment is a bone implant such as a knee or hip implant where a gap has formed between the implant and bone tissue and pain develops. More particularly, in this form of the invention, elastomeric plug 600 is inserted in a minimally invasive manner (e.g., such as using a push rod 615 to pass the elastomeric plug through a cannula to the therapy site) so as to fill the gap 620 between implant 605 and bone 610 and create a secure contact surface for the implant in order to transfer the load to the surrounding bone tissue and alleviate pain. Plugs 600 are preferably formed out of an elastomeric polymer.

In another embodiment, the plugs 600 are coated or impregnated with antibiotic or other medicants that could reduce or resolve infections.

In another embodiment, the plugs 600 are coated or impregnated with bone growth promoters or other bone healing medicants that could improve bone quality.

In another embodiment, the plugs 600 are composed of materials that resorb and are replaced with new bone growth.

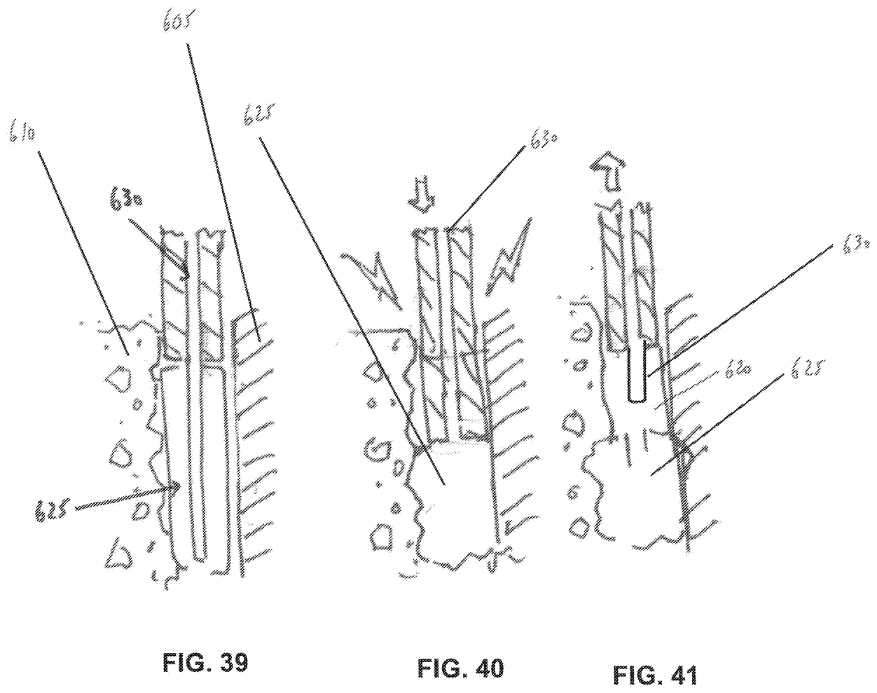

In another embodiment, and looking now at FIGS. 39-41, the invention may be implemented using a mass of biocompatible polymeric material 625 which is solid at body temperature and flowable when heated (e.g., material 625 may be a thermoplastic polymer). In this form of the invention, the mass of biocompatible polymeric material 625 is carried to the boundary region between the implant and bone on a centrally located heating core 630 which is positioned inside of the polymer mass. Heating core 630 supplies the heat necessary to transform biocompatible polymeric material 625 from a solid state to a flowable state. Note that by positioning heating core 630 within transform biocompatible polymeric material 625, the heating core is insulated from direct heat exposure to the tissue by the polymeric material. The polymeric material is preferably in the form of a polymer sleeve insert. The polymer sleeve insert 625 is stripped off the heater core 630 once the polymer sleeve insert is in a flowable state. The polymer then disperses into the gap 620 between the bone tissue and implant so as to form a layer that resolves the loosened state of the implant. In particular, a preferred embodiment is a bone implant such as a knee or hip implant where the implant has formed a gap between the metal implant and bone tissue and develops symptoms of pain.

In another embodiment, the polymer is coated or impregnated with antibiotic or other medicants that could reduce or resolve infections.

In another embodiment, the polymer is coated or impregnated with bone growth promoter or other bone healing medicants that could improve bone quality.

In another embodiment, the polymers are composed of materials that resorb and are replaced with new bone growth.

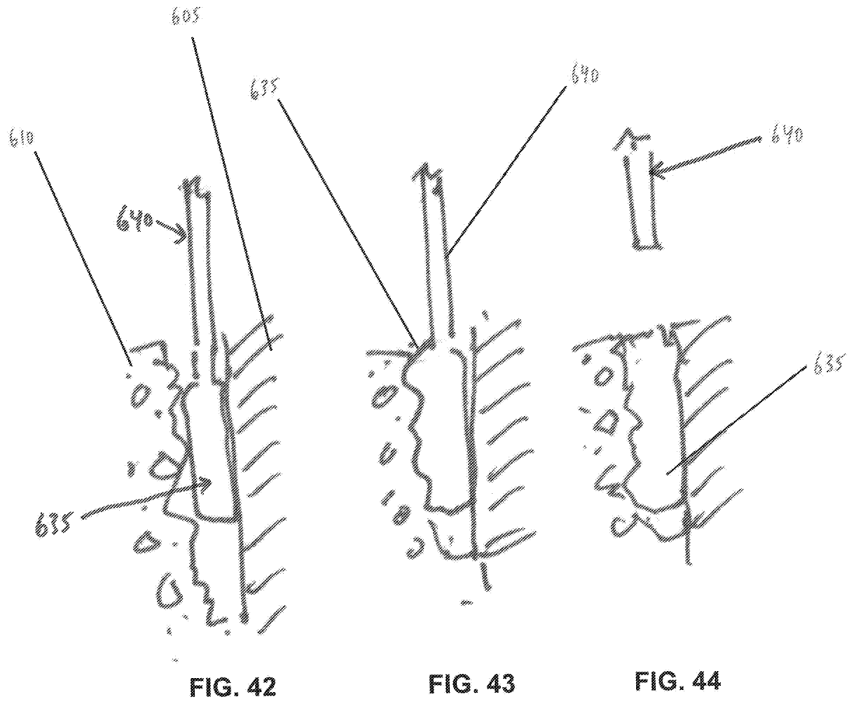

In another embodiment, and looking now at FIGS. 42-44, the present invention may be implemented using an expandable balloon 635 which is releasably attached to an insertion tool 640 for delivery to the interface location of an implant that has become loosened and detached from the tissue. In particular, a preferred embodiment is a bone implant such as a knee or hip implant where the implant has formed a gap between the metal implant and bone tissue and develops symptoms of pain. The expandable balloon 635 is advanced into the boundary region between the implant and the tissue where a gap or void has formed. Once in position, the balloon is filled with material so that it is expanded to form an inter-positional fixation of the implant to the bone (i.e., via an interference fit between the implant and bone) which allows transfer of the forces that are present in typical implant to bone interfaces. After the balloon has been positioned in the boundary region and inflated, the balloon is detached from insertion tool 640 so that the insertion tool may be removed, leaving the expandable balloon in position within the boundary region, whereby to stabilize the implant. Note that the material inserted into the balloon to cause it to expand may be a gas or liquid or flowable, settable material. In one preferred form of the invention, the balloon is filled with a thermoplastic polymer which is pumped into the balloon while it is at a heated, flowable condition, and which thereafter cools and solidifies in situ.

### Use of the Present Invention in Conjunction with Surgical Planning and Navigation Systems

It will be appreciated by those skilled in the art that the present invention may be used in conjunction with surgical planning and navigation systems. By way of example but not limitation, voids between a loosened implant and bone may be identified using imaging modalities of the sort known in the art, and then those voids may be targeted, using surgical and navigation systems of the sort known in the art, so that bone fixation material can be deployed in those voids and effect refixation of loosened implants.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

1. A method for the refixation of a loosened implant to bone, the method comprising:
providing access to a boundary region between the implant and the bone;
removing abnormal interface tissue, wear debris and/or bone cement debris from the boundary region; and
inserting a thermoplastic polymer into the boundary region so that the thermoplastic polymer engages the implant and the bone and thereby effects refixation of the loosened implant to the bone.

2. A method according to claim 1 wherein the thermoplastic polymer is inserted into the boundary region by heating the thermoplastic polymer to a flowable state, flowing the thermoplastic polymer into the boundary region, and cooling the thermoplastic polymer to a solid state.

3. A method according to claim 1 wherein the thermoplastic polymer comprises an adhesive polymer.

4. A method according to claim 1 wherein the thermoplastic polymer comprises a non-adhesive polymer.

5. A method according to claim 1 wherein providing access to the boundary region is effected by advancing a cannula to the boundary region, and further wherein the thermoplastic polymer is inserted into the boundary region through the cannula.

6. A method according to claim 1 wherein removing abnormal interface tissue, wear debris and/or bone cement debris from the boundary region is effected by at least one of lavage and mechanical debridement.

7.-11. (canceled)

12. A method for the refixation of a loosened implant to bone, the method comprising:
providing access to a boundary region between the implant and the bone;
removing abnormal interface tissue, wear debris and/or bone cement debris from the boundary region;
inserting a balloon into the boundary region; and
inflating the balloon so that the inflated balloon engages the implant and the bone and thereby effects refixation of the loosened implant to the bone.

13. A method according to claim 12 wherein providing access to the boundary region is effected by advancing a cannula to the boundary region, and further wherein the balloon is inserted into the boundary region through the cannula.

14. A method according to claim 12 wherein removing abnormal interface tissue, wear debris and/or bone cement debris from the boundary region is effected by at least one of lavage and mechanical debridement.

15.-17. (canceled)

18. A method for the refixation of a loosened implant to bone, the method comprising:
advancing a balloon cannula into a boundary region between the implant and the bone;
inflating the balloon of the balloon cannula so that the balloon engages surrounding bone, stabilizing the balloon relative to the bone and sealing the perimeter of the cannula to the surrounding bone; and
using the balloon cannula, inserting bone fixation material into the boundary region so that the bone fixation material engages the implant and the bone and thereby effects refixation of the loosened implant to the bone.

19. A method according to claim 18 wherein the balloon cannula is used to remove abnormal interface tissue, wear debris and/or bone cement debris from the boundary region before inserting the bone fixation material into the boundary region.

20. A method according to claim 19 wherein removing abnormal interface tissue, wear debris and/or bone cement debris from the boundary region is effected by at least one of lavage and mechanical debridement.

21.-23. (canceled)
